SEQUENCE LISTING (11 sequences)

SEQ ID NO: 1 - protein, 123 aa, Homo Sapiens
EVQLVESGGGLVQPGGSLRLSCAASGFTFSSSWMNWVRQAPGKGLEWVGRIYPGDGDTNYNVKFKGRFTISRDDSKNSLYLQMNSLKTHEEDTAVYYCARSGFITTVRDFDYWGQGTLVTVSS

SEQ ID NO: 2 - protein, 5 aa, Homo sapiens
SSWMN

SEQ ID NO: 3 - protein, 17 aa, Homo sapiens
RIYPGDGDTNYNVKFKG

SEQ ID NO: 4 - protein, 12 aa, Homo sapiens
SGFITTVRDFDY

SEQ ID NO: 5 - protein, 111 aa, Homo sapiens
EIVLTQSPDFQSVTPKEKVTITCRASESVDTFGISFINWFQQKPDQSPKLLIHEASNQGSGVPSRFSGSGSGTDFTLTINSLEAEDAATYYCQQTKEVPFTFGGGTKVEIK

SEQ ID NO: 6 - protein, 15 aa, Homo sapiens
RASESVDTFGISFMN

SEQ ID NO: 7 - protein, 7 aa, Homo sapiens
EASNQGS

SEQ ID NO: 8 - protein, 9 aa, Homo sapiens
QQSKEVPET

SEQ ID NO: 9 - protein, 556 aa, Homo sapiens [sequence omitted]

SEQ ID NO: 10 - DNA, 97 bp, Homo sapiens
tccctttcccaggggaggggctgggtttacgttgggagaacttttacggtgaaccaggaggttctcccaacgtaagcccagcccctcccctctgcct

SEQ ID NO: 11 - DNA, 22 bp, Homo sapiens
guucucccaacguaagcccagc

(19) United States
(12) Patent Application Publication
(10) Pub. No.: US 2015/0252431 A1
(43) Pub. Date: Sep. 10, 2015

(54) COMPOSITIONS AND METHODS FOR IDENTIFYING B CELL MALIGNANCIES RESPONSIVE TO B CELL DEPLETING THERAPY

(71) Applicant: MEDLMMUNE, LLC, GAITHERSBURG, MD (US)

(72) Inventors: YIHONG YAO, GAITHERSBURG, MD (US); KATIE STREICHER, GAITHERSBURG, MD (US); KOUSTUBH RANADE, GAITHERSBURG, MD (US); PHILIP Z. BROHAWN, GAITHERSBURG, MD (US); MICHAEL KUZIORA, GAITHERSBURG, MD (US)

(21) Appl. No.: 14/638,765

(22) Filed: Mar. 4, 2015

Related U.S. Application Data
(60) Provisional application No. 61/947,755, filed on Mar. 4, 2014.

(51) Int. Cl. C12Q 1/68; C07K 16/30

(52) U.S. Cl. CPC C12Q 1/6886; C07K 16/3061; C12Q 2600/178; C12Q 2600/106; C12Q 2600/158; C07K 2317/56; C07K 2317/24; C07K 2317/732

(57) ABSTRACT

The invention provides compositions and methods featuring the use of miR-629 for identifying subjects responsive to B-cell depleting therapies (e.g., treatment with an anti-CD19 antibody). In other embodiments, the invention features the use of miR-629 to identify subjects as having a B cell malignancy.

### CROSS-REFERENCE TO RELATED APPLICATIONS

This patent application claims the benefit of U.S. Provisional Patent Application No. 61/947,755, filed Mar. 4, 2014, which is incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 8,585 Byte ASCII (Text) file named “MR629-100US1SequenceListing.TXT,” created on Mar. 4, 2015.

### BACKGROUND OF THE INVENTION

The majority of human leukemias and lymphomas, including acute lymphoblastic leukemia (ALL), chronic lymphocytic leukemia (CLL) and non-Hodgkin lymphoma (NHL), are of B-cell origin. Therapeutic approaches based on B cell depletion by targeting B cell-restricted surface antigens with monoclonal antibodies (mAbs) have gained increasing attention. Human cluster of differentiation (CD) antigen 19 is a B cell-specific surface antigen and an attractive target for therapeutic monoclonal antibody (mAb) approaches to treat malignancies of B cell origin. An affinity optimized and afucosylated CD19 monoclonal antibody with enhanced antibody-dependent cellular cytotoxicity (ADCC) has been shown to have potent antitumour activity in preclinical models of B cell malignancies.

There is growing recognition that B cell malignancies arise from a variety of pathogenic mechanisms and that methods of characterizing these malignancies at a molecular level is useful for stratifying patients, thereby quickly directing them to effective therapies. Improved methods for predicting the responsiveness of subjects having B cell malignancies are urgently required.

### SUMMARY OF THE INVENTION

As described below, the present invention features compositions and methods featuring the use of miR-629 for identifying subjects responsive to B-cell depleting therapies (e.g., treatment with an anti-CD19 antibody). In other embodiments, the invention features the use of miR-629 to identify subjects having a B cell malignancy.

In one aspect, the invention generally provides a method of selecting therapy for a subject (e.g., human) having a B cell malignancy, the method involving detecting decreased miR-629 expression in a blood sample of the subject relative to a reference level, where detection of said decrease selects the subject for anti-CD19 antibody therapy.

In another aspect, the invention provides a method of identifying a subject as having a B cell malignancy that is responsive to treatment with an anti-CD19 antibody, the method involving detecting decreased miR-629 expression in a blood sample of the subject relative to a reference level, where detection of said decrease identifies the subject as responsive to anti-CD19 antibody treatment.

In another aspect, the invention provides a method of selecting therapy for a subject having a B cell malignancy, the method involving detecting by quantitative PCR or miRNA microarray analysis decreased miR-629 expression in a blood sample of the subject relative to a reference level, where detection of said decrease selects the subject for anti-CD19 antibody therapy.

In yet another aspect, the invention provides a method of identifying a subject as having a B cell malignancy that is responsive to treatment with an anti-CD19 antibody, the method involving detecting by quantitative PCR or miRNA microarray analysis decreased miR-629 expression in a blood sample of the subject relative to a reference level, where detection of said decrease identifies the subject as responsive to anti-CD19 antibody treatment.

In still another aspect, the invention provides a method of treating a subject selected as having a B cell malignancy responsive to treatment with an anti-CD19 antibody, the method involving administering to a selected subject an effective amount of an anti-CD19 antibody, where the subject is selected by detecting decreased miR-629 expression in a blood sample of the subject relative to a reference level.

In another aspect, the invention provides a method of administering a drug to a subject having a B cell malignancy, where the subject is identified as having a B cell malignancy responsive to treatment with an anti-CD19 antibody by detecting decreased miR-629 expression in a blood sample of the subject relative to a reference level.

In yet another aspect, the invention provides a method of depleting B cells in a subject having a B cell malignancy, the method involving detecting decreased miR-629 expression in a blood sample of the subject relative to a reference level, where detection of said decrease identifies the subject as responsive to anti-CD19 antibody therapy; and administering to the subject an anti-CD19 antibody, thereby depleting B cells in the subject.

In still another aspect, the invention provides a kit containing a primer or probe that specifically binds miR-629. In one embodiment, the kit further contains directions for the use of the kit to select or identify a subject as responsive to anti-CD19 antibody therapy.

In another aspect, the invention provides a kit containing an anti-CD19 antibody and a primer or probe that specifically binds miR-629. In one embodiment, the kit further contains directions for the use of the kit to select or identify a subject as responsive to anti-CD19 antibody therapy.

In yet another aspect, the invention provides a method of inducing or increasing anti-CD19 antibody responsiveness in a subject identified as having a B cell malignancy, the method involving administering to the subject an effective amount of an inhibitory nucleic acid molecule that targets miR-629.

In yet another aspect, the invention provides a method of depleting B cells in a subject, the method involving administering to the subject an effective amount of an inhibitory nucleic acid molecule that targets miR-629 in combination with an anti-CD19 antibody, thereby depleting B cells in the subject.

In yet another aspect, the invention provides a composition comprising an inhibitory nucleic acid molecule that targets miR-629 in combination with an anti-CD19 antibody.

In yet another aspect, the invention provides a method of identifying a subject as having a B cell malignancy, the method comprising detecting increased miR-629 expression in a blood sample of the subject relative to a reference level, where detection of said increase identifies the subject as having a B cell malignancy.

In yet another aspect, the invention provides a method of identifying a subject as having a B cell malignancy, the method comprising detecting by quantitative PCR or miRNA microarray analysis increased miR-629 expression in a blood sample of the subject relative to a reference level, where detection of said increase identifies the subject as having a B cell malignancy. In one embodiment, the reference level is the level of miR-629 expression present in a blood sample of a healthy control subject.

In yet another aspect, the invention provides an in vitro method of selecting therapy for a subject having a B cell malignancy, the method comprising detecting decreased miR-629 expression in a blood sample of the subject relative to a reference level, wherein detection of said decrease selects the subject for anti-CD19 antibody therapy.

In yet another aspect, the invention provides an in vitro method of identifying a subject as having a B cell malignancy that is responsive to treatment with an anti-CD19 antibody, the method comprising detecting decreased miR-629 expression in a blood sample of the subject relative to a reference level, wherein detection of said decrease identifies the subject as responsive to anti-CD19 antibody treatment.

In yet another aspect, the invention provides an in vitro method of selecting therapy for a subject having a B cell malignancy, the method comprising detecting by quantitative PCR or miRNA microarray analysis decreased miR-629 expression in a blood sample of the subject relative to a reference level, wherein detection of said decrease selects the subject for anti-CD19 antibody therapy.

In yet another aspect, the invention provides an in vitro method of identifying a subject as having a B cell malignancy that is responsive to treatment with an anti-CD19 antibody, the method comprising detecting by quantitative PCR or miRNA microarray analysis decreased miR-629 expression in a blood sample of the subject relative to a reference level, wherein detection of said decrease identifies the subject as responsive to anti-CD19 antibody treatment.

In another aspect, the invention provides for the use of an anti-CD19 antibody in the manufacture of a medicament for treating a subject selected in an in vitro method as having a B cell malignancy responsive to treatment with an anti-CD19 antibody, wherein the subject is selected by detecting decreased miR-629 expression in a blood sample of the subject relative to a reference level. In one embodiment, the anti-CD19 antibody is a human, humanized or chimeric antibody. In another embodiment, the anti-CD19 antibody is hypofucosylated or afucosylated. In yet another embodiment, the anti-CD19 antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 6, a light chain CDR2 comprising the amino acid sequence of SEQ ID NO: 7, and a light chain CDR3 comprising the amino acid sequence of SEQ ID NO: 8. In yet another embodiment, the anti-CD19 antibody comprises a VH domain comprising the amino acid sequence of SEQ ID NO: 1. In yet another embodiment, the anti-CD19 antibody comprises a VL domain comprising the amino acid sequence of SEQ ID NO: 5. In yet another embodiment, the anti-CD19 antibody comprises a VH domain comprising the amino acid sequence of SEQ ID NO: 1 and a VL domain comprising the amino acid sequence of SEQ ID NO: 5.

In another aspect, the invention provides for the use of an anti-CD19 antibody in the manufacture of a medicament for depleting B cells in a subject having a B cell malignancy, where the subject is selected for treatment in an in vitro method that involves detecting decreased miR-629 expression in a blood sample of the subject relative to a reference level, wherein detection of said decrease identifies the subject as responsive to anti-CD19 antibody therapy. In one embodiment, the anti-CD19 antibody is a human, humanized or chimeric antibody. In another embodiment, the anti-CD19 antibody is hypofucosylated or afucosylated. In yet another embodiment, the anti-CD19 antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 6, a light chain CDR2 comprising the amino acid sequence of SEQ ID NO: 7, and a light chain CDR3 comprising the amino acid sequence of SEQ ID NO: 8. In yet another embodiment, the anti-CD19 antibody comprises a VH domain comprising the amino acid sequence of SEQ ID NO: 1. In yet another embodiment, the anti-CD19 antibody comprises a VL domain comprising the amino acid sequence of SEQ ID NO: 5. In yet another embodiment, the anti-CD19 antibody comprises a VH domain comprising the amino acid sequence of SEQ ID NO: 1 and a VL domain comprising the amino acid sequence of SEQ ID NO: 5.

In another aspect, the invention provides for the use of an inhibitory nucleic acid molecule that targets miR-629 in the manufacture of a medicament for the treatment of a subject identified as having a B cell malignancy.

In another aspect, the invention provides for the use of an inhibitory nucleic acid molecule that targets miR-629 in the manufacture of a medicament for depleting B cells in a subject. In one embodiment, the inhibitory nucleic acid molecule is an antisense nucleic acid molecule, siRNA, or shRNA.

In another aspect, the invention provides for the use of an inhibitory nucleic acid molecule that targets miR-629 in combination with an anti-CD19 antibody in the manufacture of a medicament for treating a subject identified as having a B cell malignancy.

In another aspect, the invention provides an in vitro method of identifying a subject as having a B cell malignancy, the method involving detecting increased miR-629 expression in a blood sample of the subject relative to a reference level, wherein detection of said increase identifies the subject as having a B cell malignancy.

In another aspect, the invention provides an in vitro method of identifying a subject as having a B cell malignancy, the method comprising detecting by quantitative PCR or miRNA microarray analysis increased miR-629 expression in a blood sample of the subject relative to a reference level, wherein detection of said increase identifies the subject as having a B cell malignancy.

In various embodiments of any of the above aspects or any other aspect of the invention delineated herein, the reference level is obtained by comparing the level of miR-629 expression to the expression level of other microRNAs present in the sample; determining the range of miR-629 expression in samples obtained from a subject having a B cell malignancy that is not responsive to treatment with an anti-CD19 antibody; or by measuring the level or range of miR-629 expression in a subject or cell line having reduced sensitivity to anti-CD19 antibody treatment, resistant to the anti-proliferative effects of chemotherapy, or resistant to chemotherapy-induced apoptosis. In other embodiments of the above aspects, the reference level is obtained by measuring the fold change in expression of miR-629 using the Delta-Delta Ct method. In other embodiments of the above aspects, the reference level is obtained by measuring the range or level of miR-629 expression in a population of subjects. In various embodiments of any of the above aspects, the subject has a lymphoma or leukemia of B cell origin (e.g., non-Hodgkin's lymphoma, diffuse large B cell lymphoma, follicular lymphoma, mantle cell lymphoma, multiple myeloma, or chronic lymphocytic leukemia). In other embodiments of the above aspects, miR-629 expression is about 3 to 5-fold lower in a blood sample obtained from a subject that has responsive follicular lymphoma relative to a subject that has non-responsive follicular lymphoma. In other embodiments of the above aspects, miR-629 expression is about 5 to 7-fold lower in a subject having responsive diffuse large B-cell lymphoma relative to a subject having non-responsive diffuse large B-cell lymphoma. In other embodiments of the above aspects, the blood sample is whole blood, a peripheral blood mononucleated cell (PBMC) sample, serum, or plasma. In other embodiments of the above aspects, the anti-CD19 antibody is a human, humanized or chimeric antibody. In other embodiments of the above aspects, the anti-CD19 antibody is hypofucosylated or afucosylated. In still other embodiments of the above aspects, the anti-CD19 antibody contains a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 6, a light chain CDR2 comprising the amino acid sequence of SEQ ID NO: 7, and a light chain CDR3 comprising the amino acid sequence of SEQ ID NO: 8. In other embodiments of the above aspects, the anti-CD19 antibody contains a VH domain comprising the amino acid sequence of SEQ ID NO: 1. In other embodiments of the above aspects, the anti-CD19 antibody contains a VL domain comprising the amino acid sequence of SEQ ID NO: 5. In other embodiments of the above aspects, the anti-CD19 antibody contains a VH domain comprising the amino acid sequence of SEQ ID NO: 1 and a VL domain comprising the amino acid sequence of SEQ ID NO: 5. In other embodiments of the above aspects, the anti-CD19 antibody is MEDI-551. In other embodiments of the above aspects, the inhibitory nucleic acid molecule is an antisense nucleic acid molecule, siRNA, or shRNA. In other embodiments of the above aspects, the inhibitory nucleic acid molecule is administered prior to or concurrently with the anti-CD19 antibody.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “B cell malignancy” includes any malignancy that is derived from a cell of the B cell lineage.

By “CD19” is meant an antigen of about 90 kDa that binds an anti-CD19 antibody or fragment thereof. CD19 is found on B-lineage cells from the stem cell stage through terminal differentiation into plasma cells. In preferred embodiments, the CD19 antigen targeted by the antibodies disclosed herein (e.g., MEDI-551) is the human CD19 antigen. The sequence of one exemplary CD19 antigen is provided at GenBank Accession No. AAA69966, and shown below in SEQ ID NO. 9:

```
1 mppprllffl lfltpmevrp eeplvvkvee gdnavlqclk gtsdgptqql twsresplkp 61
    flklslglpg lgihmrplas wlfifnvsqq mggfylcqpg ppsekawqpg wtvnvegsge 121
    lfrwnvsdlg glgcglknrs segpsspsgk lmspklyvwa kdrpeiwege ppcvpprdsl 181
    nqslsqdltm apgstlwlsc gvppdsysrg plswthvhpk gpksllslel kddrpardmw 241
    vmetglllpr ataqdagkyy chrgnltmsf hleitarpvl whwllrtggw kvsavtlayl 301
    ifclcslvgi lhlqralvlr rkrkrmtdpt rrffkvtppp gsgpqnqygn vlslptptsg 361
    lgraqrwaag lggtapsygn pssdvqadga lgsrsppgvg peeeegegye epdseedsef 421
    yendsnlgqd qlsqdgsgye npedeplgpe dedsfsnaes yenedeeltq pvartmdfls 481
    phgsawdpsr eatslgsqsy edmrgilyaa pqlrsirgqp gpnheedads yenmdnpdgp 541
    dpawggggrm gtwstr
```

By “anti-CD19 antibody” is meant an antibody or fragment thereof that specifically binds a CD19 antigen. In one embodiment, an anti-CD19 antibody comprises a VH domain comprising the amino acid sequence of SEQ ID NO: 1 and a VL domain comprising the amino acid sequence of SEQ ID NO: 5.

By “miR-629” is meant a microRNA having or comprising the following sequence (SEQ ID NO 10) (prior to processing):

1 tccctttccc aggggagggg ctgggtttac gttgggagaa cttttacggt gaaccaggag 61
gttctcccaa cgtaagccca gcccctcccc tctgcct. (NCBI Accession No. NR_030714).

In another embodiment, a mature miR-629 microRNA has or comprises the following sequence SEQ ID NO. 11:

61-guucucccaacguaagcccagc-82
(miRBase Accession No. MIMAT0003298).

The function and/or expression of miR-629 can be inhibited, for example, with miRIDIAN microRNA hsa-miR-629-3p haripin inhibitor, which is commercially available from ThermoScientific.

By “delta CT method” is meant determining the Delta-Ct of each lymphoma/leukemia patient sample, which is calculated as the threshold cycle (Ct) value of miR-629 minus the mean Ct value of four housekeeping genes (RNU48, RNU24, U6, and U47). The average Delta-Ct value for all normal individuals (calculated as described for cancer patient samples) is then subtracted from the individual Delta-Ct value for each patient sample to generate a Delta-Delta-Ct value for each lymphoma/leukemia patient sample. This is related to fold change by the following equation: Fold change=2̂-(Delta-Delta-Ct).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “depletion” of B cells is meant a reduction in circulating B cells and/or B cells in particular tissue(s) relative to a baseline level. In particular embodiments, the depletion is by at least about 25%, 40%, 50%, 65%, 70%, 75%, 80%, 85%, 90%, 95% or more (e.g., 96%, 97%, 98%, or 99%) relative to the level present in the subject prior to treatment (e.g., treatment with an anti-CD19 antibody). In one particular embodiment, virtually all detectable B cells are depleted from the circulation and/or particular tissue(s).

“Detect” refers to identifying the presence, absence or amount of the analyte to be detected. In one embodiment, the analyte is miR-629.

By “miR-629 inhibitory nucleic acid molecule” is meant a double-stranded RNA, siRNA, shRNA, or antisense RNA, or a portion thereof, or a mimetic thereof, that when administered to a mammalian cell results in a decrease in the expression of miR-629. Typically, a nucleic acid inhibitor comprises at least a portion of a target nucleic acid molecule, or an ortholog thereof, or comprises at least a portion of the complementary strand of a target nucleic acid molecule. In one embodiment, a miR-629 inhibitory nucleic acid molecule inhibits at least about 10%, 25%, 50%, 75%, or even 90-100% of the miR-629 expression in the cell.

By “reference” is meant a standard of comparison. In one embodiment, a reference level is the level of miR-629 expression in a whole blood sample obtained from a healthy control subject or obtained from a subject with a B cell malignancy that is not responsive to anti-CD19 antibody treatment.

By “miR-629 siRNA” is meant a double stranded RNA capable of reducing miR-629 expression in a target cell. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream to reduce the expression of a miR-629 nucleic acid molecule.

By “specifically binds” is meant an antibody, primer, or probe that recognizes and binds a polypeptide or nucleic acid molecule of the invention, but which does not substantially recognize and bind other molecules in a sample, for example, a biological sample, which naturally includes a polypeptide or polynucleotide of the invention. In one embodiment, an anti-CD19 antibody is one that specifically binds a CD19 polypeptide. Exemplary anti-CD19 antibodies are known in the art and described herein below.

By “subject” is meant a mammal, including, but not limited to, a human or non-human mammal, such as a bovine, equine, canine, ovine, feline, or murine.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. In one embodiment, treatment of a B cell malignancy results in B cell depletion, in reducing or stabilizing the growth or proliferation of a tumor in a subject, in increasing the cell death of a malignant cell, or increasing patient survival. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### BRIEF DESCRIPTION OF THE DRAWINGS

FIG. 1A is a graph showing decreasing miR-629 expression in diffuse large B-cell lymphoma (DLBCL) cell lines with high sensitivity to anti-CD19 antibody treatment relative to cell lines having low sensitivity to anti-CD19 antibody.

FIG. 1B is a graph showing expression intensity miR signature in cell lines showing high and low sensitivity to anti-CD19 antibody administration. FIG. 1B shows that miR-629 is significantly lower in DLBCL cell lines with high sensitivity to anti-CD19 antibody treatment.

FIG. 2 is a scatter plot showing that miR-629 expression is lower in diffuse large B-cell lymphoma patients showing a complete or partial response (CR/PR) to treatment with an anti-CD19 antibody vs. non-responders with progressive disease (PD).

FIG. 3 is a scatter plot showing that miR-629 expression was lower in whole blood samples obtained from follicular lymphoma patients that responded to anti-CD19 antibody treatment (CR/PR) than in follicular lymphoma non-responders (PD).

FIG. 4 is a scatter plot showing that miR-629 expression was lower in whole blood samples from chronic lymphocytic leukemia patients that responded to anti-CD19 antibody treatment (CR/PR) than in non-responders.

FIGS. 5A and 5B are scatter plots showing miR-629 expression measured in whole blood obtained from chronic lymphocytic leukemia patients prior to treatment. The patients' response to Rituximab-ICE therapy (FIG. 5B) vs. anti-CD19 antibody-ICE therapy (FIG. 5A) was characterized.

FIGS. 5C and 5D are scatter plots showing the expression intensity miR signature in cell lines that display high and low sensitivity to anti-CD19 antibody (MEDI-551) or Rituximab treatment, respectively.

FIG. 5E is a scatter plot showing that baseline miR-629 expression is lower in DLBCL patients that respond to anti-CD19 antibody (MEDI-551) and Chemo. This effect was not observed with Rituximab. This data was obtained from patients treated at all doses (2 mg/kg and 4 mg/kg of MEDI-551 and 375 mg/m² of Rituximab).

FIG. 5F is a scatter plot showing that baseline miR-629 expression is lower in DLBCL patients that respond to anti-CD19 antibody (MEDI-551) and chemotherapy. Chemotherapy was either ICE or DHAP administered as follows: ICE will be administered via IV infusion as follows: ifosfamide 5 g/m² continuously for 24 hours with mesna on Day 2; carboplatin AUC=5 mg/mL× min (800 mg maximum) on Day 2; etoposide 100 mg/m² on Days 1, 2, and 3) in 21-day cycles. DHAP will be administered via IV infusion as follows: dexamethasone 40 mg on Days 1, 2, 3, and 4; cisplatin 100 mg/m² continuously for 24 hours on Day 1 of dosing cycle; cytarabine 2 g/m² in 3-hour infusion repeated after 12 hours (2 doses) on Day 2 in 21-day cycles. This data was obtained from patients treated with 2 mg/kg anti-CD19 antibody (MEDI-551).

FIGS. 6A-6C are scatter plots. FIGS. 6A and 6B show that miR-629 expression levels were similar pre- and post-treatment in DLBCL patients that responded to anti-CD19 antibody (CR/PR) (FIGS. 6A and 6B). FIG. 6C shows that miR-629 expression levels increased following treatment in DLBCL patients with progressive disease (PD).

FIGS. 7A and 7B are scatter plots showing that miR-629 is higher in patients with lymphoma (diffuse large B-cell lymphoma & follicular lymphoma) compared to healthy volunteers. FIG. 7A shows results obtained using miRNA microarray analysis. FIG. 7B shows results obtained using TaqMan quantitative PCR.

FIG. 8 is a scatter plot showing miR-629 expression in the specified cell types.

FIGS. 9A-9C relate to miR-629 over expression. FIG. 9A shows a miR-629/GFP expression vector. FIG. 9B is a micrograph showing GFP expression in cells expressing the miR-629/GFP expression vector. FIG. 9C is a graph showing expression of miR-629 in the DLBCL cell line Karpas-422. Following transduction of a lentiviral miR-629 expression vector, Karpas-422 cells were sorted using GFP expression into two groups, a low miR-629 group and a high miR-629 group. miR-629 expression in increased in both groups, but is higher in the group with increased GFP expression.

FIGS. 10A and 10B are graphs showing caspase activation in miR-629 over-expressing Karpas-422 lymphoma cells that were treated with 5 μM or 10 μM etoposide relative to untreated control cells. miR-629 over-expression protected Karpas-422 lymphoma cells from chemotherapy (etoposide)-induced apoptosis. Multiple clones of miR-629 over-expressing cells were generated. As the expression of miR-629 increased, a greater protection from chemotherapy (etoposide)-induced apoptosis is observed.

FIGS. 11A and 11B are graphs showing the results of cell proliferation assays in Karpas-422 lymphoma cells over-expressing miR-629 that were treated with etoposide relative to control cells transfected with vector alone (Scramble). miR-629 expression protected the cells from chemotherapy (etoposide)-induced loss of cell proliferation. As above, multiple clones of miR-629 over-expressing cells were generated. As the expression of miR-629 increased, a greater protection from chemotherapy (etoposide)-induced loss of proliferation is observed.

FIGS. 12A and 12B are graphs. FIG. 12A shows results of in vitro Antibody-Dependent Cellular Cytotoxicity (ADCC) assays in Karpas 422 cells expressing miR-629 at low or high levels relative to control cells expressing the vector alone. The ADCC results are significant because they demonstrate a shift in the ADCC response to MEDI-551 as miR-629 levels increased. Without wishing to be tied to theory, these results indicate that it is likely that miR-629 has a direct role in mediating the response to MEDI-551. FIG. 12B shows spontaneous lactate dehydrogenase (LDH) release in cells expressing low or high levels of miR-629. LDH release has been a known prognostic factor in lymphoma for many years and is measured routinely in clinical practice. These results show that miR-629 increased LDH release in lymphoma cell lines. Therefore, it is likely that miR-629 expression levels are related to the aggressiveness of the tumor. This could, in part, explain the correlation between miR-629 and response to MEDI-551.

FIG. 13 shows a logistic regression analysis of response to treatment with anti-CD19 antibody (MEDI-551) in patients with chronic lymphocytic leukemia (CLL). Points represent responders (top) and non-responders (bottom). The data show that miRNA signature expression is a potential predictive biomarker of MEDI-551 response in CLL

FIGS. 14A-D are graphs showing the results of an antibody dependent cytotoxicity (ADCC) assay. miR-629 was overexpressed in the specified cell type, and the cells were then treated with an anti-CD19 antibody (MEDI551).

FIGS. 15A and 15B are graphs showing CD19 (FIG. 15A) and CD20 (FIG. 15B) expression assayed using an Allophycocyanin (APC)-conjugated secondary antibody in nine cell lines that varied in their sensitivity to anti-CD19 antibody (MEDI551) treatment. Mean fluorescent intensity (MFI) ratio was measured in control transfected cells, miR transfected cells, and non-transfected cells. Neither CD19 nor CD20 changed following miR-629 over-expression.

FIG. 16 is a graph showing that miR-629 expression levels (fold change compared to normal blood) in baseline blood samples from DLBCL patients does not correlate with a miRNA expression signature in blood shown previously to predict increased patient survival following treatment with the chemotherapeutic combination including Rituximab, Cyclophosphamide, Hydroxydaunomycin (or doxorubicin), vincristine also termed (ONCOVIN®), and Prednisolone (R-CHOP) (Alencar, et al., Clin Cancer Res; 17(12) Jun. 15, 2011). The R-CHOP response-associated miRNA signature does not correlate with MEDI-551 response-associated miRNA signature in DLBCL Blood.

FIG. 17 is a graph showing the miR-629 was present in exosomes isolated from cells that stably over-express miR-629. In fact, miR-629 was present at 12-20 fold higher levels in these exosomes.

FIGS. 18A and 18B are graphs showing a preliminary analysis of the effect of miR-629 nucleofection on natural killer (NK) cells. miR-629 expression increased following nucleofection (FIG. 18A); and expression of genes in cytolytic pathways and related natural killer cell activation/adhesion pathways was reduced by 40-60%. Genes analyzed include granzyme B (GZMB), GZMA, GZMM, cathepsin D (CTSD), perforin 1 (PRF1), CD63, CD96, and interferon regulatory factor 7.

### DESCRIPTION OF ANTI-CD19 (16C4) ANTIBODY SEQUENCES

VH domain SEQ ID NO: 1:
Glu Val Gln Leu Val Glu Ser Gly Gly Gly Leu Val
Gln Pro Gly Gly Ser Leu Arg Leu Ser Cys Ala Ala
Ser Gly Phe Thr Phe Ser Ser Ser Trp Met Asn Trp
Val Arg Gln Ala Pro Gly Lys Gly Leu Glu Trp Val
Gly Arg Ile Tyr Pro Gly Asp Gly Asp Thr Asn Tyr
Asn Val Lys Phe Lys Gly Arg Phe Thr Ile Ser Arg
Asp Asp Ser Lys Asn Ser Leu Tyr Leu Gln Met Asn
Ser Leu Lys The Glu Asp Thr Ala Val Tyr Tyr Cys
Ala Arg Ser Gly Phe Ile Thr Thr Val Arg Asp Phe
Asp Tyr Trp Gly Gln Gly Thr Leu Val Thr Val Ser
Ser
VH CDR1 SEQ ID NO: 2:
SSWMN
VH CDR2 SEQ ID NO: 3:
RIYPGDGDTNYNVKFKG
VH CDR3 SEQ ID NO: 4:
SGFITTVRDFDY
VL domain SEQ ID NO: 5:
Glu Ile Val Leu Thr Gln Ser Pro Asp Phe Gln Ser
Val Thr Pro Lys Glu Lys Val Thr Ile Thr Cys Arg
Ala Ser Glu Ser Val Asp Thr Phe Gly Ile Ser Phe
Ile Asn Trp Phe Gln Gln Lys Pro Asp Gln Ser Pro
Lys Leu Leu Ile His Glu Ala Ser Asn Gln Gly Ser
Gly Val Pro Ser Arg Phe Ser Gly Ser Gly Ser Gly
Thr Asp Phe Thr Leu Thr Ile Asn Ser Leu Glu Ala
Glu Asp Ala Ala Thr Tyr Tyr Cys Gln Gln Thr Lys
Glu Val Pro Phe Thr Phe Gly Gly Gly Thr Lys Val
Glu Ile Lys
VL CDR1 SEQ ID NO: 6:
RASESVDTFGISFMN
VL CDR2 SEQ. ID NO: 7:
EASNQGS
VL CDR3 SEQ ID NO: 8:
QQSKEVPET

### DETAILED DESCRIPTION OF THE INVENTION

The invention provides compositions and methods featuring the use of miR-629 for identifying subjects responsive to B-cell depleting therapies (e.g., treatment with an anti-CD19 antibody).

The invention is based, at least in part, on the discovery that miR-629 expression in blood samples of subjects with B cell malignancies can be used to characterize the subject's responsiveness to anti-CD19 antibody treatment. As reported in detail below, a number of human non-Hodgkin B cell lymphoma cell lines were identified as having high or low sensitivity to anti-CD19 antibody treatment using an in vitro antibody-dependent cellular cytotoxicity (ADCC) assay. miR-629 expression levels were reduced in blood samples obtained from diffuse large B-cell lymphoma subjects that were responsive to anti-CD19 antibody treatment. miR-629 expression levels were also reduced in blood samples obtained from follicular lymphoma subjects and chronic lymphocytic leukemia subjects responsive to anti-CD19 antibody treatment.

Accordingly, the invention provides methods for identifying subjects that have a B cell malignancy that is likely to respond to anti-CD19 antibody treatment based on the level of miR-629 expression in a subject blood sample.

### Types of Biological Samples

In characterizing the responsiveness of a B cell malignancy in a subject to anti-CD19 antibody treatment, the level of miR-629 expression is measured in different types of biologic samples. In one embodiment, the biologic sample is a blood, serum, or plasma sample. In one preferred embodiment, the biological sample is a blood sample comprising peripheral blood mononuclear cells, lymphocytes, and monocytes.

miR-629 expression may be at least about 3 to 5-fold lower or about 5 to 7-fold lower in a blood sample obtained from a subject that is responsive to anti-CD19 antibody treatment than the level of expression in a non-responsive subject (e.g., a subject with progressive disease). In another embodiment, miR-629 expression is at least about 5, 10, 20, or 30-fold higher in a subject with a B cell malignancy than in a healthy control. Fold change values are determined using any method known in the art. In one embodiment, fold change is determined by calculating 2^{-ΔΔCt} using miR-629 expression in a healthy volunteer or in anti-CD19 antibody non-responsive subject

### Selection of a Treatment Method

As reported herein below, subjects suffering from a B cell malignancy may be tested for miR-629 expression in the course of selecting a treatment method. Patients characterized as having reduced miR-629 expression relative to a reference level are identified as responsive to anti-CD19 treatment.

A number of standard treatment regimens are available for the selected patients. These treatments can be used in combination with the methods of the invention. In particular embodiments, anti-CD19 treatment is administered in combination with ICE (Ifosfamide, Carboplatin and Etoposide).

### CD19

Human cluster of differentiation (CD) antigen 19 is a B cell specific antigen that belongs to the immunoglobulin domain containing superfamily of transmembrane receptors. CD19 is expressed on B cells throughout their lineage from pro-B cells to the plasma cell stage, when CD19 expression is down regulated. CD19 is not expressed on hematopoietic stem cells or on B cells before the pro-B-cell stage. Importantly, expression of CD19 is maintained following malignant transformation of B cells, and CD19 is expressed on the majority of B cell malignancies. The widespread and relatively stable expression of CD19 on B-cell malignancies makes this antigen an attractive target for mAb-based therapies.

### Anti-CD19 Antibodies

Subject's having a B-cell malignancy responsive to treatment with an anti-CD19 antibody are identified by characterizing the level of miR-629 expression present in their blood. Once selected for treatment, such subjects may be administered virtually any anti-CD19 antibody known in the art. Suitable anti-CD19 antibodies include, for example, known anti-CD19 antibodies, commercially available anti-CD19 antibodies, or anti-CD19 antibodies developed using methods well known in the art.

MEDI-551 is a CD19 mAb with potent ADCC effector function. MEDI-551 is the afucosylated form of the CD19 mAb anti-CD19-2, developed by humanization and affinity optimization of the HB12b mAb (Kansas & Tedder, 1991; Yazawa et al, 2005; Herbst et al, 2010). MEDI-551 is generated by the expression of mAb anti-CD19-2 in a fucosyltransferase-deficient producer cell line, a procedure that generates a homogenously afucosylated mAb with increased affinity to FccRIIIA and enhanced ADCC activity (Herbst et al., J Pharmacol Exp Ther, 2010. 335(1):213-222).

In certain embodiments, the methods and compositions described herein utilize the anti-CD19 antibody 16C4 (see e.g., U.S. Publication No. 2008/0138336), which is incorporated by reference, or antigen binding fragment thereof. 16C4 is a CD19 mAb that has been shown to have potent ADCC effector function. 16C4 is the afucosylated form of the CD19 mAb anti-CD19-2, which was developed by humanization and affinity optimization of the HB12b mAb (Kansas G S and Tedder T F. J Immunol, 1991; 147:4094-4102; Yazawa et al., Proc Natl Acad Sci, 2005: 102(42):15178-15183; Herbst et al., J Pharmacol Exp Ther, 2010. 335(1):213-222). 16C4 and MEDI-551 both comprise heavy chain CDRs comprising the amino acid sequence of SEQ ID NO: 2, SEQ ID NO: 3, and SEQ ID NO: 4, and light chain CDRs comprising the amino acid sequence of SEQ ID NO: 6, SEQ ID NO: 7, and SEQ ID NO: 8. The CDRs of SEQ ID NOs: 2 to 4 and SEQ ID NOs: 6 to 8 are comprised within the VH of SEQ ID NO: 1 and the VL of SEQ ID NO: 5. As such, the person skilled in the art will appreciate that antibodies comprising the CDRs of SEQ ID NOs: 2 to 4 and 6 to 8 may also be used in methods and compositions of the present invention.

The present disclosure encompasses antibodies that are derivatives of antibody 16C4 that bind to human CD19. Standard techniques known to those of skill in the art can be used to introduce mutations (e.g., additions, deletions, and/or substitutions) in the nucleotide sequence encoding an antibody, including, for example, site-directed mutagenesis and PCR-mediated mutagenesis that are routinely used to generate amino acid substitutions. In one embodiment, the VH and/or VK CDRs derivatives may include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, less than 2 amino acid substitutions, or 1 amino acid substitution relative to the original VH and/or VK CDRs of the 16C4 anti-CD19 antibody. In another embodiment, the VH and/or VK CDRs derivatives may have conservative amino acid substitutions made at one or more predicted non-essential amino acid residues (e.g., amino acid residues which are not critical for the antibody to specifically bind to human CD19). Mutations can also be introduced randomly along all or part of the VII and/or VK CDR coding sequences, such as by saturation mutagenesis, and the resultant mutants can be screened for biological activity to identify mutants that retain activity. Following mutagenesis, the encoded antibody can be expressed and the activity of the antibody can be determined. The percent identity of two amino acid sequences can be determined by any method known to one skilled in the art, including, but not limited to, BLAST protein searches.

In other embodiments, the anti-CD19 antibody is described for example in U.S. Patent Application Publications 20130330328, 20130183306, 20110104150, each of which is incorporated herein by reference in their entirety. In certain embodiments, an anti-CD19 antibody of the disclosure is a known anti-CD19 antibody including, but not limited to HD37 (IgG1, kappa) (DAKO North America, Inc., Carpinteria, Calif.), BU12 (Callard et al., J. Immunology, 148(10):2983-7 (1992)), 4G7 (IgG1) (Meeker et al., Hybridoma, 3(4):305-20 (1984 Winter)). J4.119 (Beckman Coulter, Krefeld, Germany), B43 (PharMingen, San Diego, Calif.), SJ25C1 (BD PharMingen, San Diego, Calif.), FMC63 (IgG2a) (Zola et al., Immunol. Cell. Biol. 69(PT6): 411-22 (1991); Nicholson et al., Mol. Immunol., 34:1157-1165 (1997); Pietersz et al., Cancer Immunol. Immunotherapy, 41:53-60 (1995)), 89B(B4) (IgG1) (Beckman Coulter, Miami, Fla.; Nadler et al., J. Immunol., 131:244-250 (1983)), and/or HD237 (IgG2b) (Fourth International Workshop on Human Leukocyte Differentiation Antigens, Vienna, Austria, 1989; and Pezzutto et al, J. Immunol., 138(9):2793-2799 (1987)). In other embodiments, an anti-CD19 antibody of the disclosure is any of the anti-CD19 antibodies described in U.S. Patent Application Publication Nos. 2008/0138336 and 2009/0142349 and U.S. Pat. Nos. 7,462,352 and 7,109,304. In exemplary embodiments, an anti-CD19 antibody is the 16C4 antibody, or an antigen binding fragment thereof, as described in U.S. Patent Application Publication No. 2008/0138336 and below.

Antibodies useful in the invention include immunoglobulins, monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies disclosed herein), intrabodies, and epitope-binding fragments of any of the above. In particular, antibodies include immunoglobulin molecules and immunologically active fragments of immunoglobulin molecules, e.g., molecules that contain at least one antigen-binding site.

Anti-CD19 antibodies encompass monoclonal human, humanized or chimeric anti-CD19 antibodies. Anti-CD19 antibodies used in compositions and methods of the invention can be naked antibodies, immunoconjugates or fusion proteins. In certain embodiments, an anti-CD19 antibody mediates human antibody-dependent cellular cytotoxicity (ADCC), complement-dependent cell-mediated cytotoxicity (CDC), and/or apoptosis in an amount sufficient to deplete circulating B cells.

Anti-CD19 antibodies useful in the methods of the invention reduce or deplete B cells (e.g., malignant B cells) when administered to a human. Depletion of B cells can be in circulating B cells, or in particular tissues such as, but not limited to, bone marrow, spleen, gut-associated lymphoid tissues, and/or lymph nodes. In one embodiment, anti-CD19 antibody may deplete circulating B cells, blood B cells, splenic B cells, marginal zone B cells, follicular B cells, peritoneal B cells, and/or bone marrow B cells. In one embodiment, an anti-CD19 antibody depletes progenitor B cells, early pro-B cells, late pro-B cells, large-pre-B cells, small pre-B cells, immature B cells, mature B cells, antigen stimulated B cells, and/or plasma cells. Such depletion is achieved, for example, by antibody-dependent cell-mediated cytotoxicity (ADCC), and/or by blocking of CD19 interaction with its intended ligand, and/or complement dependent cytotoxicity (CDC), inhibition of B cell proliferation and/or induction of B cell death (e.g., via apoptosis).

If desired, the anti-CD19 antibody is engineered to have enhanced ADCC activity relative to the parent antibody. Methods for creating antibody variants having enhanced ADCC activity are known in the art and described herein below. In certain embodiments, an anti-CD19 antibody is an afucosylated antibody having enhanced ADCC activity.

In certain embodiments, an anti-CD19 antibody is a human, humanized or chimeric antibody having an IgG isotype, particularly an IgG1, IgG2, IgG3, or IgG4 human isotype or any IgG1, IgG2, IgG3, or IgG4 allele found in the human population. Antibodies of the human IgG class have advantageous functional characteristics, such as a long half-life in serum and the ability to mediate various effector functions (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. The IgG1 subclass has the high ADCC activity and CDC activity in humans (Chemical Immunology, 65, 88 (1997)).

In other embodiments, an anti-CD19 antibody is an isotype switched variant of a known anti-CD19 antibody (e.g., to an IgG1 or IgG3 human isotype) such as those described above. In other embodiments, an anti-CD19 antibody immunospecifically binds to human CD19 and has a dissociation constant (K_{D}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method known to one of skill in the art (e.g., a BIAcore assay, ELISA) (Biacore International AB, Uppsala, Sweden). In other embodiments, an anti-CD19 antibody of the disclosure may immunospecifically bind to a human CD19 antigen and may have a dissociation constant (K_{D}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using a method known to one of skill in the art (e.g., a BIAcore assay, ELISA). In certain embodiments, an anti-CD19 antibody of the disclosure may immunospecifically bind to human CD19 and may have a dissociation constant (K_{D}) of 500 pM, 100 pM, 75 pM or 50 pM as assessed using a method known to one of skill in the art (e.g., a BIAcore assay, ELISA).

### Engineering Effector Function

If desired, subjects identified as responsive to anti-CD19 antibody therapy are administered anti-CD19 antibodies that are modified with respect to effector function, so as to enhance the effectiveness of the antibody in treating B cell malignancies, for example. An exemplary effector function is antibody-dependent cell-mediated cytotoxicity, or ADCC, which is a cell-mediated reaction in which non-specific cytotoxic cells recognize bound antibody on a target cell and subsequently cause lysis of the target cell. The cytotoxic cells, or effector cells, may be leukocytes which express one or more FcRs. Effector cells express at least Fc gamma RI, FC gamma RII, Fc gamma RIII and/or Fc gamma RIV in mouse. Human leukocytes that mediate ADCC include peripheral blood mononuclear cells (PBMC), natural killer (NK) cells, monocytes, cytotoxic T cells and neutrophils. Of these cells, the primary cells for mediating ADCC are NK cells, which express Fc gamma RIII Monocytes express Fc gamma RI, Fc gamma RII, Fc gamma RIII and/or Fc gamma RIV. FcR expression on hematopoietic cells is summarized in Ravetch and Kinet, Annu. Rev. Immunol., 9:457-92 (1991).

One method for enhancing the effector function of antibodies is by producing engineered glycoforms. Engineered glycoforms are generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include, but are not limited to, those described in Umana et al, 1999, Nat. Biotechnol 17:176-180: Davies et al., 2001 Biotechnol Bioeng 74:288-294; Shields et al, 2002, J Biol Chem 277:26733-26740; Shinkawa et al., 2003, J Biol Chem 278:3466-3473; U.S. Pat. No. 6,602,684; U.S. Patent Application Publication No. 2003/0157108 (U.S. application Ser. No. 10/277,370); U.S. Patent Application Publication No. 2003/0003097 (U.S. application Ser. No. 10/113,929); PCT WO 00/61739A1; PCT WO 01/292246A1; PCT WO 02/311140A1; PCT WO 02/30954A 1; Potillegent™ technology (Biowa, Inc. Princeton, N.J.); GlycoMAb™ glycosylation engineering technology (GLYCART biotechnology AG, Zurich, Switzerland), each of which is incorporated herein by reference in its entirety. See, e.g., WO 00061739; EA01229125; US 20030115614: Okazaki et al., 2004, JMB, 336: 1239-49, each of which is incorporated herein by reference in its entirety. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

An antibody can also be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. See, for example, Shields, R. L. et al. (2002) J. Biol. Chem. 277:26733-26740; Umana et al. (1999) Nat. Biotech. 17:176-1, as well as, U.S. Pat. No. 6,946,292; European Patent No: EP 1,176,195; PCT Publications WO 03/035835; and WO 99/54342 each of which is incorporated herein by reference in its entirety.

In one embodiment, an anti-CD19 comprises a variant Fc region that mediates enhanced antibody-dependent cellular cytotoxicity (ADCC). In one embodiment, an anti-CD19 antibody comprises an Fc region having complex N-glycoside-linked sugar chains linked to Asn297 in which fucose is not bound to N-acetylglucosamine in the reducing end, wherein said Fc region mediates enhanced antibody-dependent cellular cytotoxicity (ADCC).

In vitro assays known in the art and described herein can be used to determine whether anti-CD19 antibodies used in compositions and methods of the disclosure are capable of mediating ADCC. Exemplary assays are described in U.S. Pat. No. 5,500,362 or U.S. Pat. No. 5,821,337. Notably, useful effector cells for such assays include peripheral blood mononuclear cells (PBMC) and Natural Killer (NK) cells. Alternatively, or additionally, ADCC activity of the molecules of interest may be assessed in vivo. e.g., in an animal model such as that disclosed in Clynes et al. (Proc. Natl. Acad. Sci. (USA), 95:652-656 (1998)). The assay may also be performed using a commercially available kit, e.g. CytoTox 96™ (Promega).

### B Cell Malignancies

B cell malignancies are characterized by the pathological expansion of specific B cell subsets, for example, precursor B cell acute lymphoblastic leukemia is characterized by an abnormal expansion of B cells corresponding to pro-B cell/Pre-B cell developmental stages. The malignant B cells maintain cell surface expression of normal B cell markers, such as CD19. An anti-CD19 antibody may therefore deplete malignant B cells in a human subject.

A therapy comprising anti-CD19 antibodies as described herein, can be used to treat B cell diseases, including B cell malignancies. Exemplary B cell malignancies include, but are not limited to: B cell subtype non-Hodgkin's lymphoma (NHL) including low grade/follicular NHL, small lymphocytic (SL) NHL, intermediate grade/follicular NHL, intermediate grade diffuse NHL, high grade immunoblastic NHL, high grade lymphoblastic NHL, high grade small non-cleaved cell NHL; mantle-cell lymphoma, and bulky disease NHL; Burkitt's lymphoma; multiple myeloma; pre-B acute lymphoblastic leukemia and other malignancies that derive from early B cell precursors; common acute lymphocytic leukemia (ALL); chronic lymphocytic leukemia (CLL) including immunoglobulin-mutated CLL and immunoglobulin-unmutated CLL; hairy cell leukemia; Null-acute lymphoblastic leukemia; Waldenstrom's Macroglobulinemia; diffuse large B cell lymphoma (DLBCL) including germinal center B cell-like (GCB) DLBCL, activated B cell-like (ABC) DLBCL, and type 3 DLBCL; pro-lymphocytic leukemia; light chain disease; plasmacytoma; osteosclerotic mycloma; plasma cell leukemia; monoclonal gammopathy of undetermined significance (MGUS); smoldering multiple myeloma (SMM); indolent multiple myeloma (IMM); Hodgkin's lymphoma including classical and nodular lymphocyte pre-dominant type: lymphoplasmacytic lymphoma (LPL); and marginal-zone lymphoma including gastric mucosal-associated lymphoid tissue (MALT) lymphoma.

Treatment of relapses of these cancers is also contemplated. Lymphocyte-predominant Hodgkins disease (LPHD) is a type of Hodgkin's disease that tends to relapse frequently despite radiation or chemotherapy treatment. Chronic lymphocytic leukemia is one of four major types of leukemia. A cancer of mature B-cells called lymphocytes, chronic lymphocytic leukemia is manifested by progressive accumulation of cells in blood, bone marrow and lymphatic tissues. Indolent lymphoma is a slow-growing, incurable disease in which the average subject survives between six and 10 years following numerous periods of remission and relapse.

The desired level of B cell depletion will depend on the disease. In one embodiment, the depletion of the B cells, which are the target of the anti-CD19 antibodies is sufficient to reduce or eliminate progression of the disease. Disease progression is assessed by a physician, for example, by monitoring tumor growth (size), proliferation of the cancerous cell type, metastasis, and/or by monitoring other signs and symptoms of the particular cancer. In one embodiment, the B cell depletion is sufficient to reduce or eliminate progression of disease for at least about 2, 3, 4, 5, or 6 months. In other embodiments, the B cell depletion is sufficient to increase the time in remission by at least about 6, 9, or 12 months, or even by about 2, 3, 4, or 5 years. In another embodiment, the B cell depletion is sufficient to cure the disease. In certain embodiments, the B cell depletion in a cancer subject reduces the number or level of malignant B cells by at least about 50%, 75%, 80%, 85%, 90%, 95%, 99% or even 100% of the baseline level before treatment.

The parameters for assessing efficacy or success of treatment of the neoplasm will be known to the physician (e.g., oncologist). Generally, the physician will look for a reduction in disease progression, an increased time in remission, the presence of stable disease. For B cell neoplasms, measurable criteria may include, e.g., time to disease progression, an increase in duration of overall and/or progression-free survival. In the case of leukemia, a bone marrow biopsy can be conducted to determine the degree of remission. Complete remission can be defined as the leukemia cells making up less than 5 percent of all cells found in a subject's bone marrow 30 days following treatment.

The following references describe lymphomas and chronic lymphocytic leukemia, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D: Chronic Lymphocytic Leukemia. Chap. 72, pp 1350-1362, in: Hematology, Basic Principles and Practice, 3rd ed. Hoffman et al. (editors). Churchill Livingstone, Philadelphia, 2000.

### Kits

The invention provides kits for characterizing the responsiveness of a subject to anti-CD19 antibody treatment.

In one embodiment, the kit includes a therapeutic composition containing an effective amount of an antibody that specifically binds a CD19 polypeptide in unit dosage form.

A diagnostic kit of the invention provides a reagent (e.g., TaqMan primers/probes for both miR-629 and housekeeping reference genes) for measuring relative expression of miR-629.

In some embodiments, the kit comprises a sterile container which contains a therapeutic or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In one embodiment, a kit of the invention comprises reagents for measuring miR-629 expression and an anti-CD19 antibody. If desired, the kit further comprises instructions for measuring miR-629 expression and/or instructions for administering the anti-CD19 antibody to a subject having a B cell malignancy, e.g., a malignancy selected as responsive to anti-CD19 antibody treatment. In particular embodiments, the instructions include at least one of the following: description of the therapeutic agent; dosage schedule and administration for treatment or prevention of B cell malignancy or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

### Inhibitory Nucleic Acids

Inhibitory nucleic acid molecules are those oligonucleotides that inhibit the expression of a nucleic acid molecule or polypeptide. As reported in detail below, the invention provides methods for identifying a B cell malignancy in a subject that is responsive to treatment with an anti-CD19 antibody by measuring miR-629 expression in a blood sample, where detection of a decrease in miR-629 expression relative to a reference identifies the subject as having a B cell malignancy that is responsive to anti-CD19 antibody treatment. In view of this discovery, it is likely that methods that reduce the expression of miR-629 in the subject would induce or enhance anti-CD19 antibody responsiveness in the subject.

Accordingly, the invention provides single and double stranded inhibitory nucleic acid molecules (e.g., DNA, RNA, and analogs thereof) that target miR-629 and reduce its expression. Exemplary inhibitory acid molecules include siRNA, shRNA, and antisense RNAs.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of miR-629, siRNAs may be designed to reduce expression of miR-629. Such siRNAs could be administered to a subject systemically to reduce miR-629 expression. 21 to 25 nucleotide siRNAs targeting miR-629 are used, for example, as therapeutics to treat a B cell malignancy.

The inhibitory nucleic acid molecules of the present invention may be employed as double-stranded RNAs for RNA interference (RNAi)-mediated knock-down of expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, each of which is hereby incorporated by reference, for a description of inducible shRNA.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### miR-629 Levels were Decreased in Anti-CD19 Antibody Sensitive Cell Lines

A number of human non-Hodgkin B cell lymphoma cell lines were identified as having high or low sensitivity to anti-CD19 antibody treatment using an in vitro Antibody-dependent cellular cytotoxicity (ADCC) assay. In particular, Karpas-422, a human B cell non-Hodgkin lymphoma, Oci-Ly-19, diffuse large cell lymphoma, SUD-HL-6, a follicular B cell lymphoma (ATCC® CRL2959™), and Toledo cell lines, a non-Hodgkin lymphoma model system, were identified as having high sensitivity to anti-CD19 antibody treatment. In contrast, DB (diffuse large cell lymphoma), ARH-77 (EBV-transformed B lymphoblastoid cell line), and RL (non-Hodgkin's lymphoma B cell line) were identified as having low sensitivity to anti-CD19 antibody treatment.

These cell lines were characterized by analyzing their microRNA expression. microRNAs/miRNAs are small single-stranded RNA molecules that inhibit translation of multiple target mRNAs. Roles for miRNA have identified in cardiovascular disease, diabetes, cancer, and other diseases. The role for miRNA in predicting response to various therapeutics is not well understood.

Interestingly, 17 miRNAs were identified that were differentially expressed between Diffuse large B-cell lymphoma (DLBCL or DLBL) cell lines of varying sensitivity to anti-CD19 antibody treatment. These differences were observed using multiple platforms, including Affymetrix miRNA microarray and TaqMan qPCR.

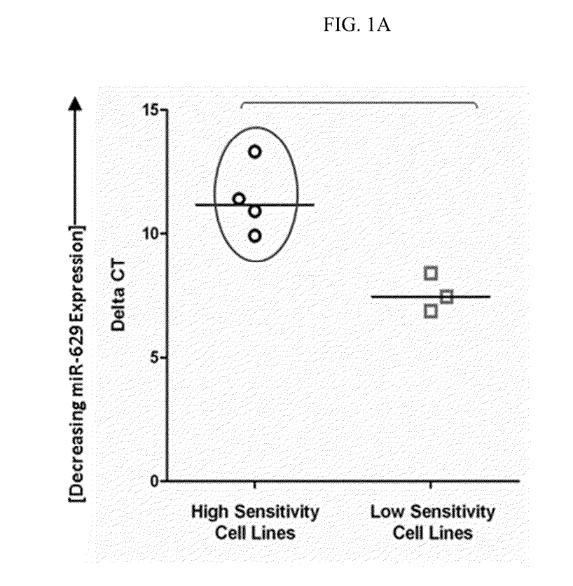

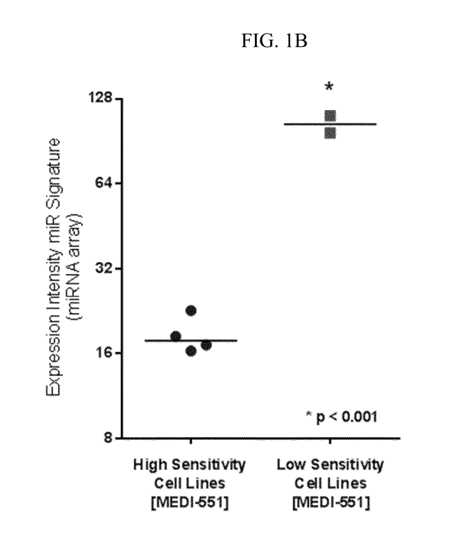

The following microRNAs had significant differences: miR-629; miR-99b; miR-let-7e; miR-15a; and miR-29a. The most significant difference was in expression of miR-629. miR-629 expression levels were significantly lower in diffuse large B-cell lymphoma cell lines with high sensitivity to anti-CD19 antibody treatment (FIGS. 1A and 1B) than cell lines having low sensitivity to anti-CD19 antibody. In determining low versus high sensitivity, the EC50s for the high sensitivity cell lines were at least 100-fold (in other cases 1000-fold or more) lower than the low sensitivity cell lines in in vitro ADCC assays.

In sum, miR-629 expression was significantly different between cell lines of high sensitivity (n=4) versus low sensitivity (n=3) to in vitro ADCC with an anti-CD19 antibody (MED-551). This effect appears to be specific to responsiveness to an anti-CD19 antibody. Alterations in miR-629 expression did not correlate with responsiveness to Rituximab.

### Example 2

### miR-629 Expression was Reduced in Diffuse Large B-Cell Lymphoma Patients Responsive to Anti-CD19 Antibody Treatment

miR-629 expression levels were measured in baseline whole blood samples from diffuse large B cell lymphoma (DLBCL), follicular lymphoma (FL), and Chronic lymphocytic leukemia (CLL) patients treated with an anti-CD19 antibody treatment as a single agent in Clinical Trial No. CP204, A Phase 1, Dose-escalation Study of MEDI-551, a Humanized Monoclonal Antibody Directed Against CD19, in Adult Subjects With Relapsed or Refractory Advanced B-Cell Malignancies.

Patients receiving anti-CD19 antibody treatment were categorized as having a complete or partial response.
CR/PR (complete or partial response): 5 diffuse large B-cell lymphoma, 6 follicular lymphoma, 3 Chronic lymphocytic leukemia
PD (progressive disease): 10 diffuse large B-cell lymphoma, 2 follicular lymphoma, and 3 Chronic lymphocytic leukemia
SD (stable disease): 4 diffuse large B-cell lymphoma, 3 follicular lymphoma, and 9 Chronic lymphocytic leukemia

miR-629 expression levels were measured in baseline peripheral blood mononucleated cell (PBMC) samples from Chronic lymphocytic leukemia patients treated with MEDI-551 or Rituximab+Bendamustine (CP-1019).
22 MEDI-551-treated patients (10 CR/PR; 3 PD; 4 SD)
12 Rituximab-treated patients (6 CR/PR; 2 PD; 2 SD)

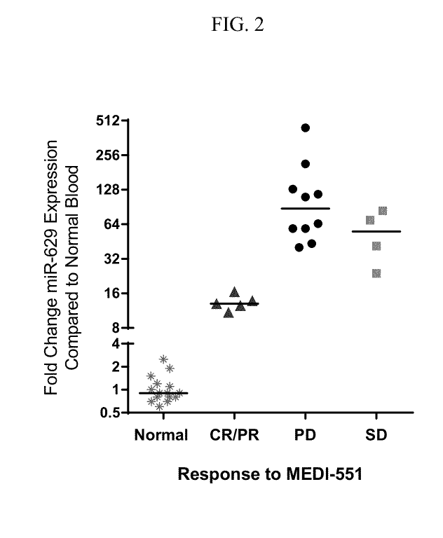

Surprisingly, miR-629 expression was significantly lower (˜7-fold) in diffuse large B-cell lymphoma patients showing a complete or partial response to treatment with an anti-CD19 antibody (CR/PR) vs. non-responders (PD) (FIG. 2). miR-629 expression was measured in whole blood samples prior to treatment with the anti-CD19 antibody. In sum, phase I clinical trial data demonstrated that baseline miR-629 expression was lower in diffuse large B cell lymphoma patients that responded to an anti-CD19 antibody (MEDI-551). Interestingly, miR-629 expression was significantly increased in samples of patients that have diffuse large B-cell lymphoma compared to levels of miR-629 present in blood samples obtained from normal control subjects.

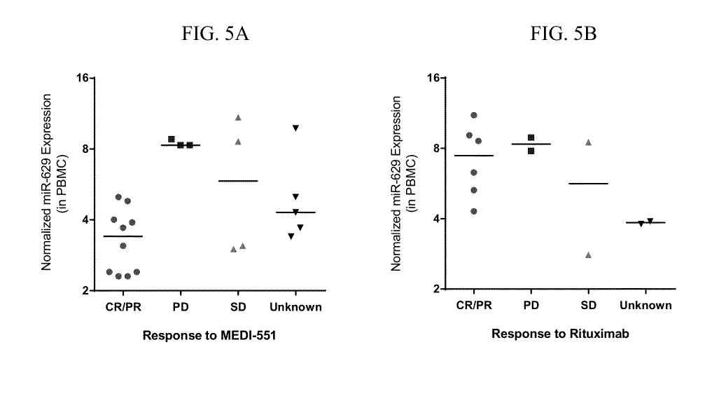

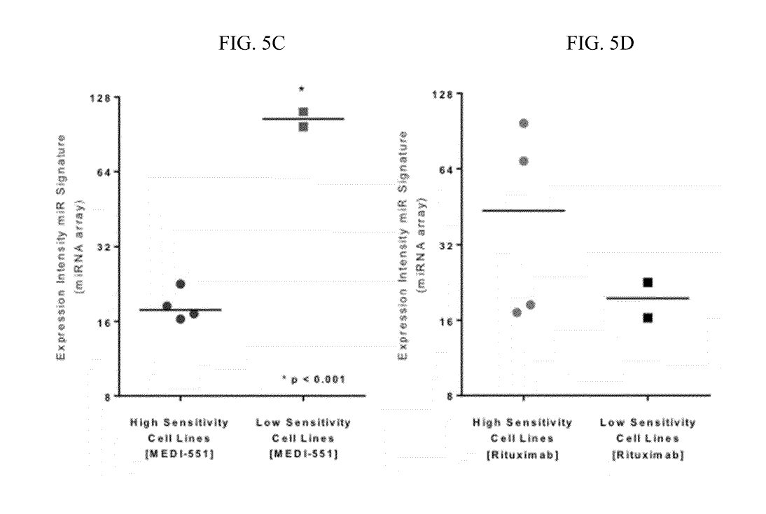

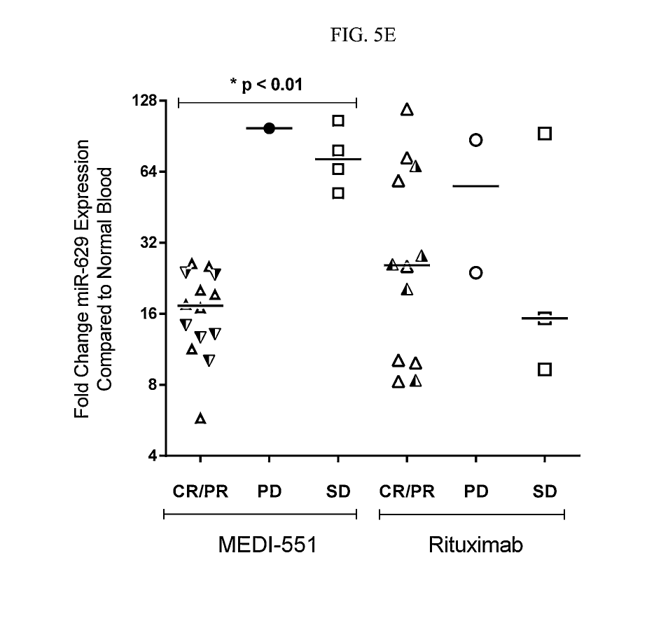

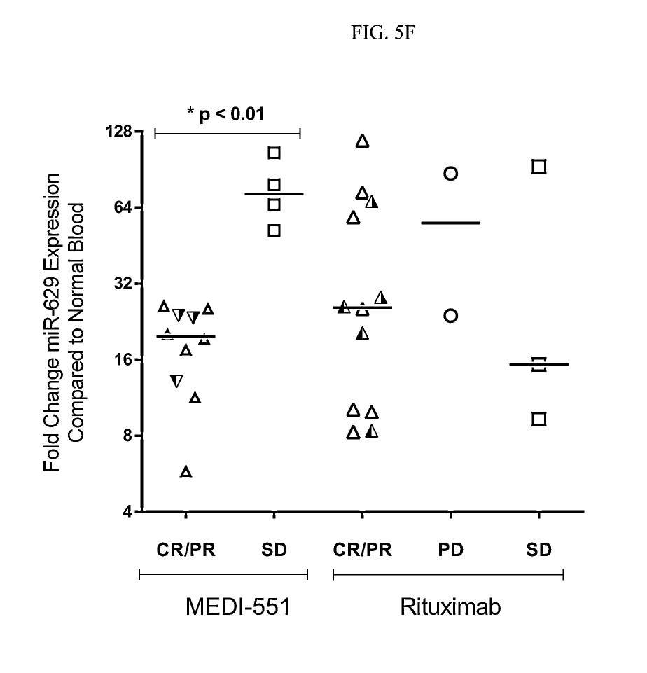

Levels of miR-629 expression has been compared between patients that are treated with either an anti-CD19 antibody (MEDI-551) plus ICE/DHAP or Rituximab plus ICE/DHAP. miR-629 levels have been characterized as increased or decreased in patients that respond to anti-CD19 antibody treatment administered in combination with ICE (Ifosfamide, Carboplatin and Etoposide)/DHAP compared to those that respond to anti-CD20 antibody therapy in combination with ICE/DHAP. These studies will also characterize any alterations in miR-629 that are specifically reduced in DLBCL patients that respond to anti-CD19 antibody, as compared to patients that respond to treatment with Rituximab. The results of the study are shown in FIG. 5.

### Example 3

### miR-629 Expression was Reduced in Follicular Lymphoma Patients Responsive to Anti-CD19 Antibody Treatment

miR-629 expression levels were measured in whole blood samples obtained from follicular lymphoma patients prior to treatment with anti-CD19 antibody. miR-629 expression is significantly increased in follicular lymphoma blood compared to normal blood.

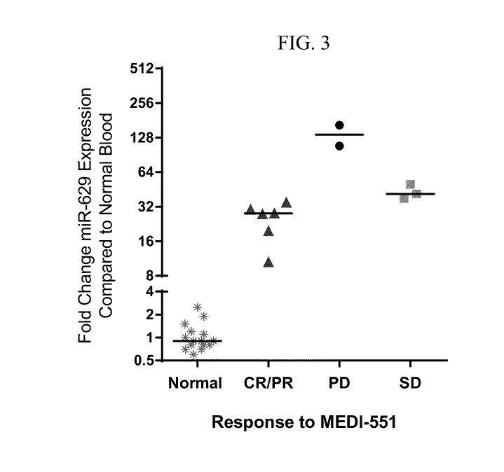

miR-629 expression was considerably lower (˜5-fold) in whole blood samples obtained from follicular lymphoma patients that responded to anti-CD19 antibody treatment (CR/PR) than in follicular lymphoma non-responders (PD) in (FIG. 3).

### Example 4

### miR-629 Expression is Increased in Chronic Lymphocytic Leukemia Patients

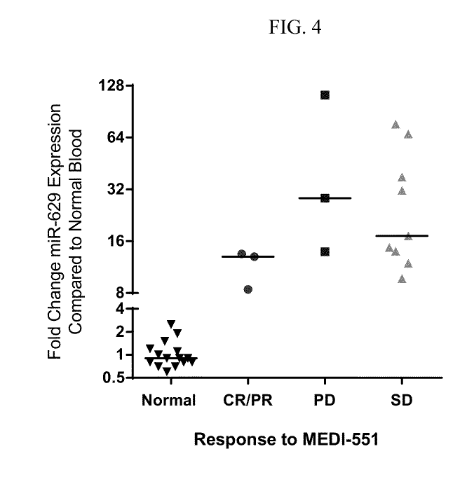

miR-629 expression was increased in whole blood obtained from patients with chronic lymphocytic leukemia (CLL) as compared to blood obtained from normal control subjects. Preliminary results appear to indicate that miR-629 expression was lower in whole blood samples obtained from chronic lymphocytic leukemia patients that responded to anti-CD19 antibody treatment (CR/PR) than in chronic lymphocytic leukemia non-responders (FIG. 4). These initial observations will be confirmed in additional patients.

### Example 5

### Preliminary Data Shows miR-629 Expression was Reduced in Chronic Lymphocytic Leukemia Patients Responsive to Anti-CD19 Antibody-ICE (Bendamustine) Treatment

miR-629 expression was measured in whole blood obtained from chronic lymphocytic leukemia patients prior to treatment. The patients' response to Rituximab-ICE therapy vs. anti-CD19 antibody-ICE therapy was characterized (FIGS. 5A and 5B). Although the sample size was small, no association between miR-629 expression levels was found between CR/PR and PD patients treated with Rituximab-ICE (FIG. 5B). In contrast, miR-629 expression levels were lower in anti-CD19 antibody-ICE responsive chronic lymphocytic leukemia patients.

### Example 6

### miR-629 Expression was Lower in Diffuse Large B Cell Lymphoma Patients Responsive to Anti-CD19 Antibody Treatment

miR-629 expression was lower in diffuse large B cell lymphoma cell lines with high sensitivity to anti-CD19 antibody (MEDI-551) (FIG. 5C). No such correlation was observed in diffuse large B cell lymphoma cell lines based on their sensitivity to Rituximab (FIG. 5D). Similar observations were made in diffuse large B cell lymphoma patients in CP1088 trial. (FIG. 5E)

miR-629 expression was measured in whole blood samples obtained from diffuse large B cell lymphoma patients prior to treatment. Nineteen patients were subsequently treated with an anti-CD19 antibody (MEDI-551) and chemotherapy (ICE or DHAP). Seventeen patients were treated with Rituximab. Interestingly, miR-629 expression was significantly lower (˜4-fold) in patients that responded to anti-CD19 antibody (MEDI-551) treatment (CR/PR) vs. non-responders (SD/PD) (FIG. 5E). This was true whether patients were treated with 2 mg/kg or 4 mg/kg of an anti-CD19 antibody (MEDI-551) (FIGS. 5E and 5F). No such correlation was observed with regard to Rituximab responsiveness (FIG. 5E).

### Example 7

### miR-629 Expression Levels Increased in Patients that Did not Respond to Anti-CD19 Antibody Treatment

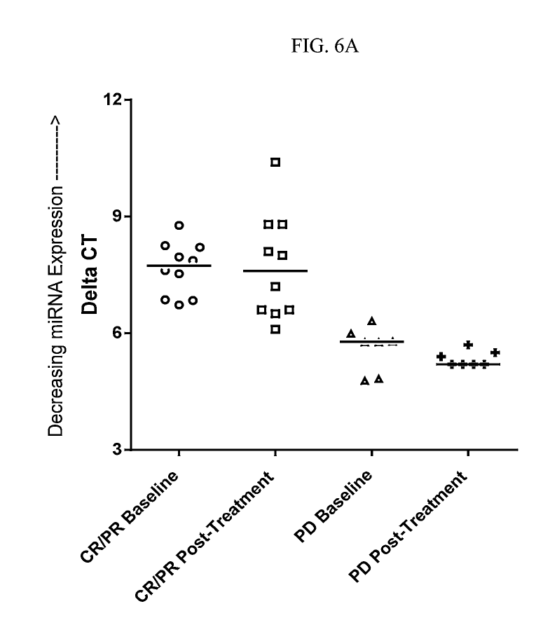

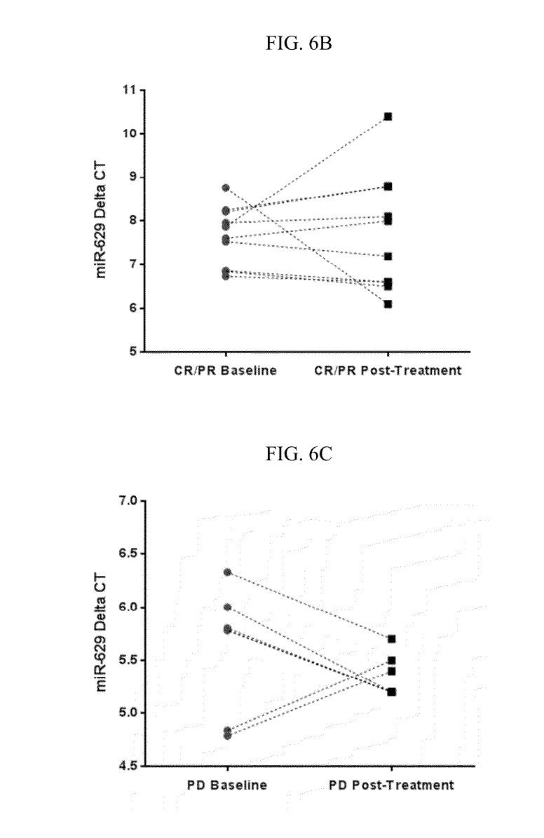

Interestingly, miR-629 expression levels were similar pre- and post-treatment in DLBCL subjects that responded to anti-CD19 antibody (CR/PR) (FIGS. 6A and 6B). In contrast, miR-629 expression levels tended to increase following treatment in patients with progressive disease (PD) (FIG. 6C). These results indicate that miR-629 plays a specific role in the responsiveness to anti-CD19 antibody (MEDI-551), such that cancer progression correlates with levels of this microRNA.

### Example 8

### miR629 Expression is Higher in Lymphoma Patients Relative to Healthy Controls

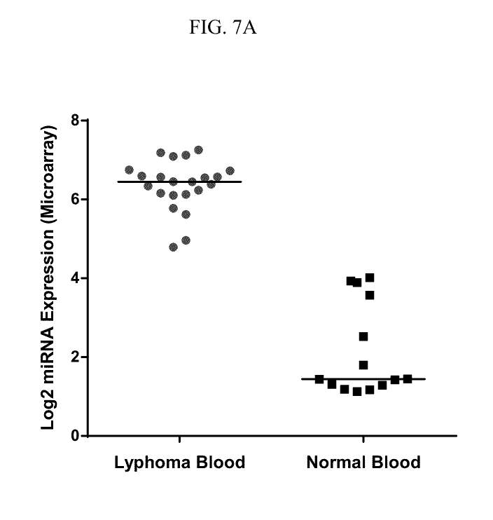

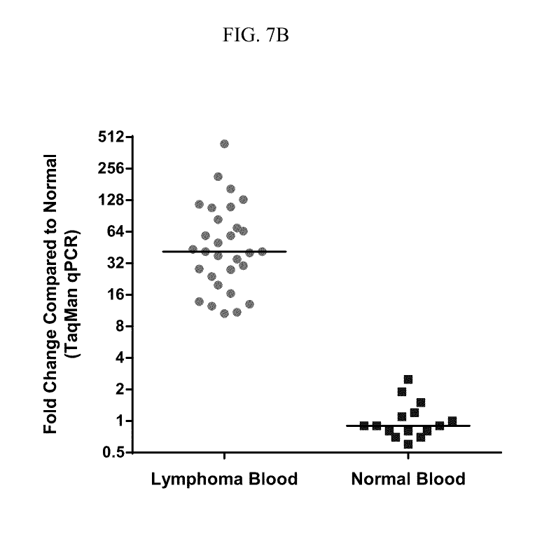

miR-629 is higher in all patients with lymphoma (diffuse large B-cell lymphoma & follicular lymphoma) compared to healthy volunteers when measured using TaqMan quantitative PCR (FIG. 7B) or using miRNA microarray analysis (FIG. 7A). In Examples 1-6, miR-629 was measured by TaqMan quantitative PCR.

The source of miR-629 in lymphoma blood is unknown. Nevertheless, it is unlikely to reflect an alteration in the number of B cells in diffuse large B-cell lymphoma, follicular lymphoma, or chronic lymphocytic leukemia. No relationship was observed between miR-629 levels and baseline B cell counts (CD19 or CD20) in diffuse large B-cell lymphoma, follicular lymphoma, or chronic lymphocytic leukemia patients.

miR-629 was expressed to a greater degree in whole blood of diffuse large B-cell lymphoma patients compared to healthy whole blood (16-fold minimum). miR-629 levels are higher in normal monocytes and B cells relative to other cell types. miR-629 expression is higher in CD14⁺ and CD19⁺ cells. These levels remain considerably lower than that observed in diffuse large B-cell lymphoma/follicular lymphoma patients (FIG. 8).

### Example 9

### miR-629 Over-Expression Protects Against Chemotherapy-Induced Apoptosis and Loss of Cell Proliferation

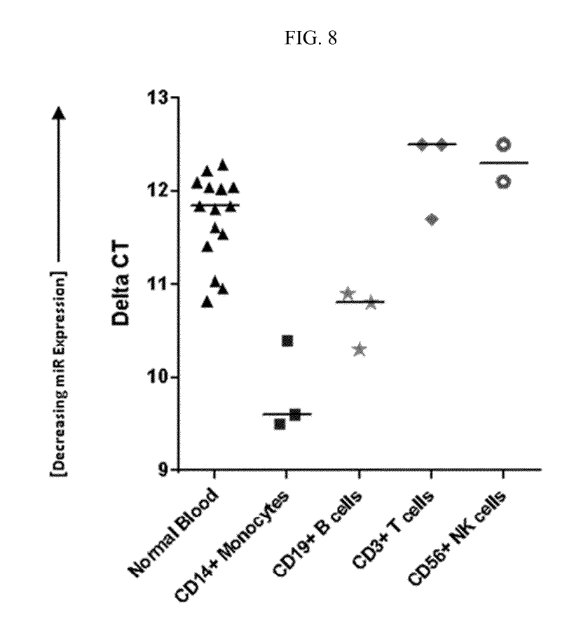

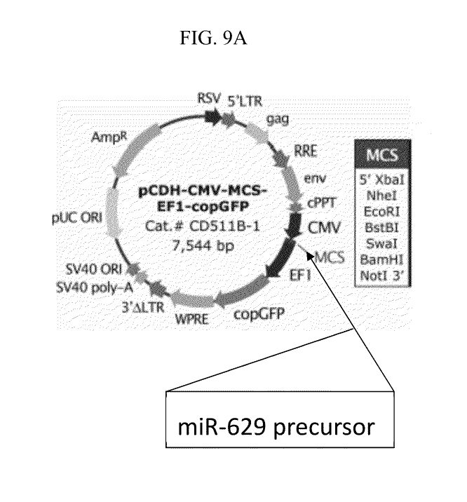

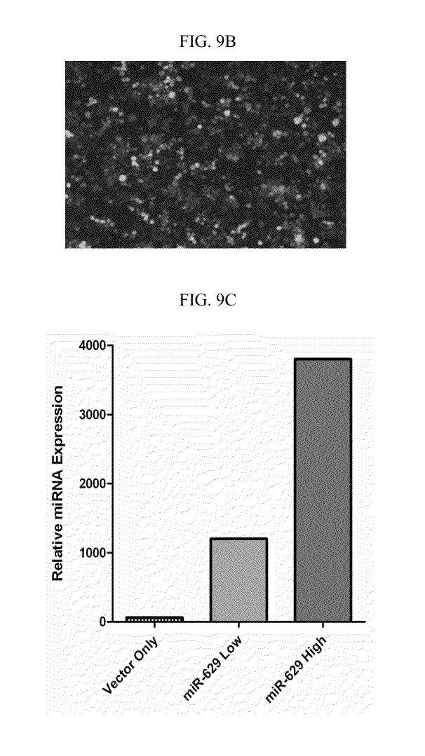

miR-629 over-expressing Karpas-422 cell lines were generated using the miR-629 expression vector shown at FIG. 9A. Karpas-422 cells are a DLBCL cell line that has low levels of miR-629 and was highly sensitive to anti-CD19 antibody (MEDI-551) in vitro ADCC. The cells also expressed a GFP reporter that provided for visual monitoring of miR-629 expression levels (FIG. 9B). Cells were transfected with the miR-629 expression vector or a control vector that did not include the miR-629 precursor insert. The cells were then sorted by FACS based on GFP expression into miR-629-high and miR-629-low expressing populations. miR-629-expressing single cell clones were also generated by limiting dilution. Relative levels of miR-629 expression are shown in FIG. 9C.

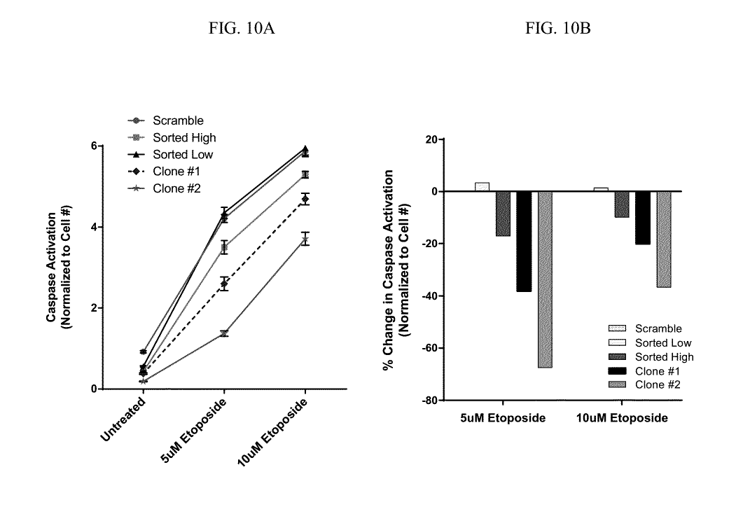

FIGS. 10A and 10B show caspase activation in miR-629 over-expressing Karpas-422 lymphoma cells. Interestingly, miR-629 over-expression protected Karpas-422 lymphoma cells from chemotherapy (etoposide)-induced apoptosis (FIGS. 10A and 10B).

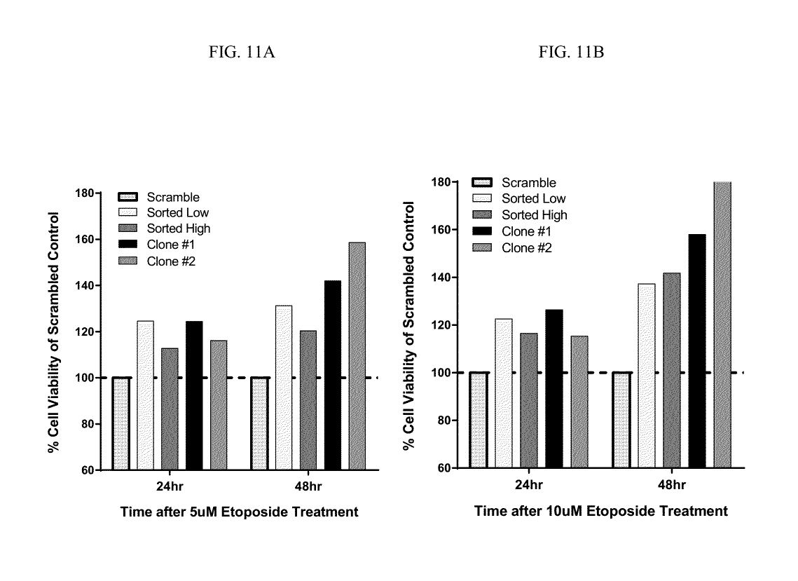

miR-629 over-expression also protected Karpas-422 lymphoma cells from chemotherapy (etoposide)-induced loss of cell proliferation (FIGS. 11A and 11B). Accordingly, methods for decreasing miR-629 levels in B cell malignancies are expected to restore the cells sensitivity to chemotherapy (i.e., the ability of chemotherapy to reduce cell proliferation and increase apoptosis).

### Example 10

### miR-629 Over-Expression Increased Spontaneous Lactate Dehydrogenase (LDH) Release

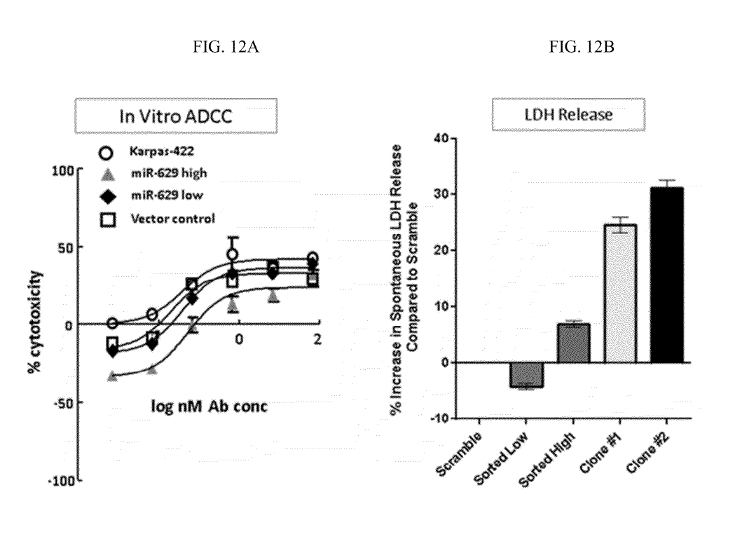

miR-629 over-expression was associated with an increase in spontaneous LDH Release in vitro and a slight shift in Ec50 for anti-CD19 antibody treatment in in vitro ADCC (FIGS. 12A and 12B).

### Example 11

### Baseline miR-629 Expression Predicts Response to MEDI-551 and Chemotherapy

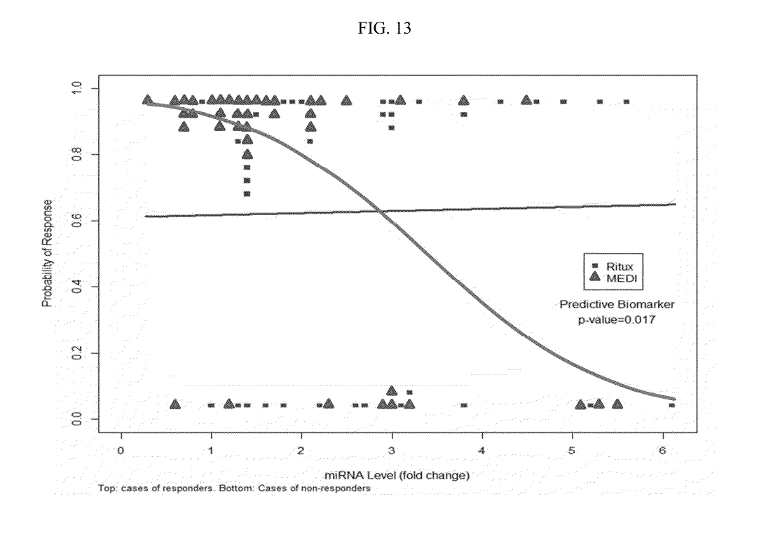

FIG. 13 provides a logistic regression analysis of response of patients treated with anti-CD19 antibody (MEDI-551) or Rituximab and miRNA signature expression (measured in pre-treatment PBMC samples and shown as fold change relative to expression in healthy volunteers). Only patients who had both miRNA data and ≧1 post-baseline disease assessment were included in the analysis. Curves shown in FIG. 13 represent the predicted probability of response across miRNA signature levels based on the regression model. The crossing of the 2 curves (indicating the treatment-by-biomarker interaction) indicates that the miRNA signature is likely to be a predictive biomarker for anti-CD19 antibody (MEDI-551)-responsiveness in chronic lymphocytic leukemia. Baseline miR-629 expression predicts response to anti-CD19 antibody (MEDI-551) and chemotherapy, but not Rituximab and chemotherapy (ICE-bendamustine).

### Example 12

### Effects of Altering miR-629 Expression

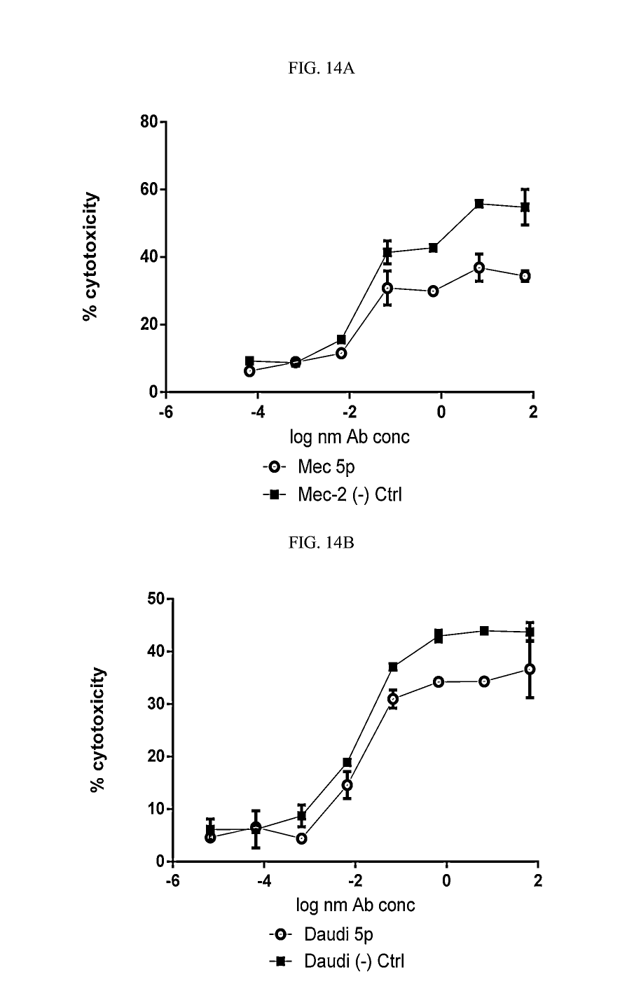

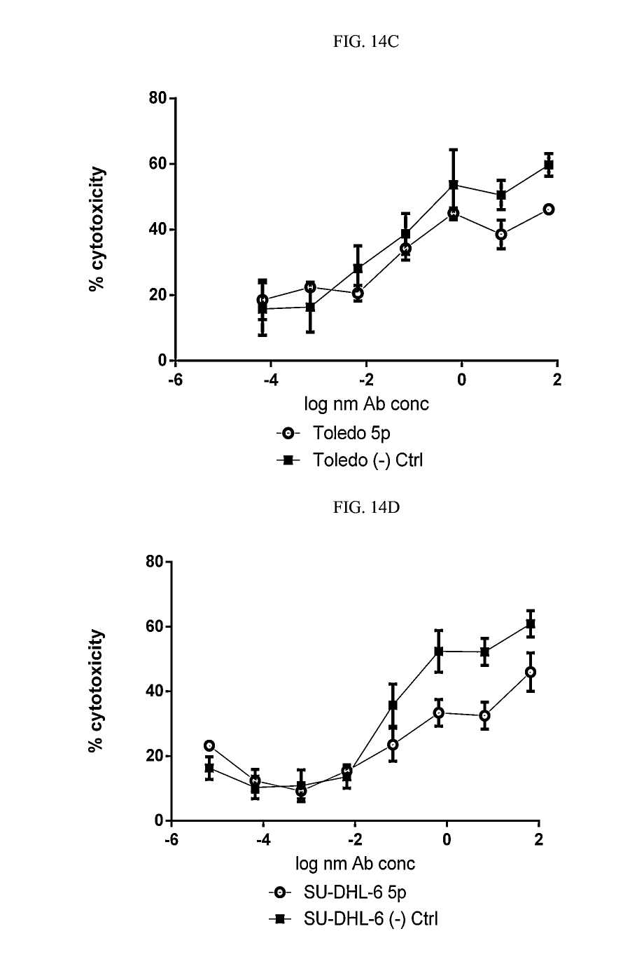

The effect of altering miR-629 on sensitivity to treatment with an anti-CD19 antibody (MEDI551) was explored. Human leukemia and lymphoma cell lines Daudi, Toledo, and RL were obtained from American Type Culture Collection (ATCC); Karpas 1106P, Karpas 422, OCI-Ly-19, MEC2 were obtained from Deutsche Sammlung von Mikroorganismen and Zellkulturen (DSMZ, Germany). Cell lines were transfected with 50 uM of a miR-629 mimic (Karpas 1106P, Karpas 422, Daudi, MEC2, OCI-Ly-19, SU-DHL-6, and Toledo), a miR-629 hairpin inhibitor (RL and ARH-77), or respective negative control oligonucleotides (all from Dharmacon) using PrimeFect (Lonza) for 24 hours. CD19 and CD20 surface expression was determined by flow cytometry (LSRII, BD Biosciences) using their respective fluorescently labeled monoclonal antibodies. Surface expression is reported as mean fluorescent intensity (MFI) and was averaged for untransfected as well as miR-629 and negative control transfected cells. When miR-629 was over-expressed, MEC-2 and Daudi cell lines showed a 15-25% reduced sensitivity to anti-CD19 antibody (MEDI551) (FIGS. 14A and 14B), while a 15-20% difference in cytotoxicity was observed in Toledo, and SU-DHL-6 cell lines (FIGS. 14C and 14D).

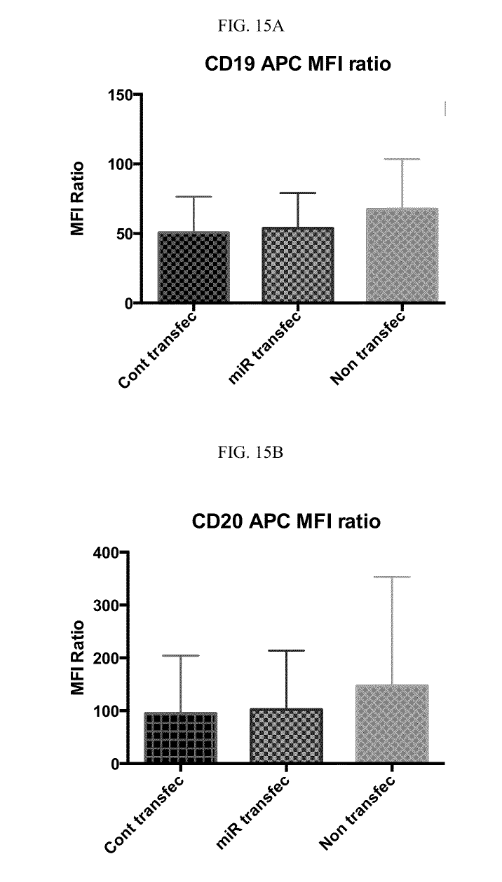

The effect of over-expressing miR-629 on CD19 and CD20 surface expression was assayed in nine cell lines that varied in their sensitivity to treatment with an anti-CD19 antibody (MEDI551) (FIGS. 15A and 15B). CD19 and CD20 expression was measured using an Allophycocyanin (APC)-conjugated antibody. Mean fluorescent intensity (MFI) ratio was measured in control transfected cells, miR transfected cells, and non-transfected cells. No alteration in CD19 or CD20 surface expression was observed in response to miR-629 over-expression. This suggests that alterations in CD19 and CD20 expression does not account for differences in the cells' sensitivity to anti-CD19 antibody (MEDI551).

### Example 13

### No Correlation Exists Between the R-CHOP microRNA Signature and the Anti-CD19 Antibody (MEDI-551) microRNA Signature (miR-629 Expression)

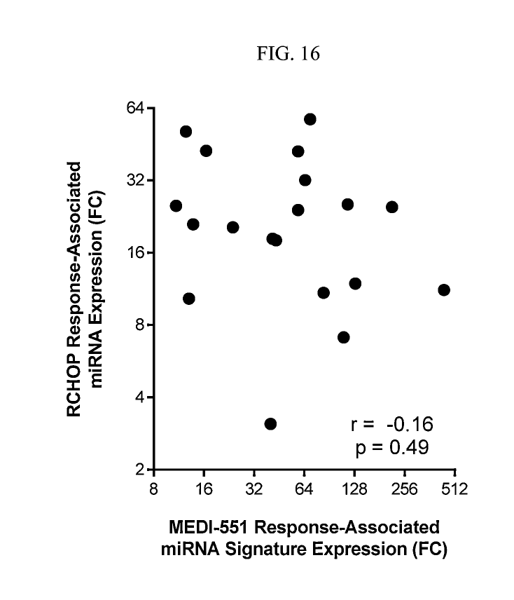

A miRNA signature was shown to predict increased survival in diffuse large B cell lymphoma patients treated with the chemotherapeutic combination R-CHOP, which includes Rituximab, Cyclophosphamide, Hydroxydaunomycin (or doxorubicin), vincristine also termed (ONCOVIN®), and Prednisolone, (Alencar et al., Clin. Cancer Res. 2011; 17:4125-35). No correlation was observed between the expression of this signature in baseline blood samples from DLBCL patients and the expression of miR-629 (FIG. 16). This result supports the specificity of the MEDI-551 response-associated miRNA signature that has been clinically observed.

### Example 14

### miR-629 was Observed in Exosomes

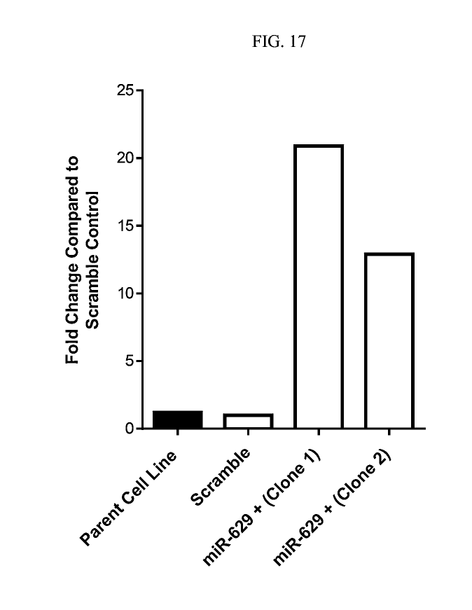

Exosomes are cell-derived vesicles that are released into biological fluids by most—if not all—cell types, including tumor cells. Evidence suggests a key role for exosome-mediated intercellular communication in processes involved in tumor development and progression. Using the Total Exosome Isolation kit (Invitrogen, cat #4478359), exosomes were isolated from supernatants of Karpas-422 cell lines stably over-expressing either mIR-629 or miRNA scrambled control. Cell culture media was harvested and spun at 2,000×g for 30 minutes to remove cells and debris. Cell-free culture media was transferred to new tubes and treated with 0.5 vol Total Exosome Isolation reagent. Culture media and reagent were mixed well by pipetting or vortexing until a homogeneous solution was achieved. Samples were incubated overnight at 4° C. Following incubation, samples were spun at 10,000×g for 1 hr at 4° C. Supernatant was aspirated and discarded, and pelleted exosomes were resuspended in 0.2 volumes Exosome Resuspension Buffer (Invitrogen, cat #4478545). Resuspended exosomes were incubated 5-10 min at room temperature. Denaturing solution was prewarmed to 37° C., and 1 volume was added to exosome suspension. The solution was incubated on ice for 10 min and then extracted using 1 vol acid:phenol:chloroform proportionate to the starting exosome sample volume. Samples were vortexed for 30-60 seconds and spun at 13,000×g for 5′ at RT. Aqueous phase was transferred to a new tube and 1.25 vol EtOH was added per sample. After thorough mixing, 700 uL sample was placed onto Zymo column (Zymo Research, ZR RNA MicroPrep, cat # R1060/R1061) and spun at 10,000×g for 15 sec. This procedure was repeated until all lysate was passed through filter. Wash I was added at 700 uL and spun as above. Wash II was performed using 500 uL, spun as above and repeated 1 X. A final spin at 10,000×g was performed for 1 min to remove residual liquid. Exosomal RNA was eluted in a fresh collection tube using 20 uL preheated (95° C.) nuclease-free water. Columns were spun at 10,000×g for 30 seconds and eluate reapplied to same column with an additional 5 uL nuclease free water and spun a second time under same conditions. Samples were qualified and semi-quantified using a Pico Agilent chip. Interestingly, miR-629 was identified in exosomes secreted by the miR-629 over-expressing cell lines. miR-629 was over-expressed by 12-20 fold in these exosomes relative to control cells (FIG. 17).

Without wishing to be bound by theory, tumor-derived miR-629 may be delivered to natural killer (NK) cells via exosomes, thereby reducing NK cell activation NK cells are granular lymphocytes that produce inflammatory cytokines and spontaneously kill target cells. Where baseline levels of miR-629 are high, this hypothesis suggests that NK cell activity would be low. An anti-CD19 antibody (MEDI551) could therefore have reduced activity through NK-cell mediated antibody dependent cytotoxicity (ADCC) and lead to poor response to treatment. Low miR-629 at baseline would not be expected to result in an increased response to Rituxan (also referred to as Rituximab) because Rituxan works through other mechanisms of action in addition to NK cell-mediated ADCC, whereas MEDI551 does not.

### Example 15

### miR-629 Over-Expression Alters NK Cell Function

The effect of miR-629 on NK cell function is assessed by analyzing the expression of genes known to be altered during NK cell activation, including cytolytic pathway genes (e.g., granzyme B (GZMB), GZMA, GZMM, cathepsin B and D, perforin 1), cell surface/adhesion molecules (e.g., CD96 (TACTILE), CD63 granulophysin), and NK cell activation receptors. NK cell function can also be assayed in an interferon-gamma or granzyme B ELISA. Granzymes are serine proteases that are released by cytoplasmic granules within cytotoxic T cells and natural killer (NK) cells. Granzymes induce programmed cell death in target cells, including cancer cells.

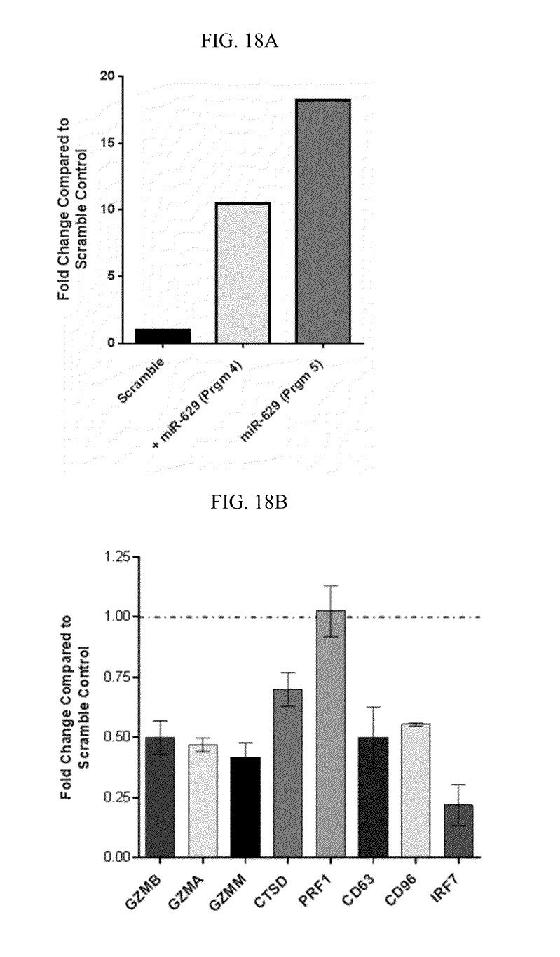

Initial results indicate that miR-629 over-expression alters NK cell function (FIGS. 18A and 18B). The effect of miR-629 over-expression on NK cell function was analyzed following miR-629 nucleofection. miR-629 levels increased following nucleofection (FIG. 18A). and this increase resulted in a 40-60% reduction in genes associated with cytolytic pathways and NK activation/adhesion. Genes analyzed include granzyme B, granzyme A, granzyme M, cathepsin D, perforin 1, CD63, CD96, and interferon regulatory factor 7 (FIG. 18B). Nucleofection was carried out using the following methodology, NK-92 cells (ATCC #CRL-2407) were maintained in Advanced RPMI (LifeTech) media containing 2 mM glutamine, 10% FBS and 10 ng/mL IL-2 (PeproTech #200-02) at a density of 0.2-1.5e6 cells/mL and sub-cultured every 3-4 days. NK-92 cells were nucleofected using the Amaxa Cell Line Nucleofector Kit R and the Amaxa Nucleofector II device (Lonza) as follows. Twelve-well tissue culture plates were prepared by filling the appropriate number of wells with 1.5 mL culture media and pre-incubating in a humidified 37° C./5% CO2 incubator. The nucleofection working solution was prepared by adding 0.45 mL Supplement to 2.05 mL Cell Line Nucleofector Solution R. For a single nucleofection, 5e6 cells were spun down at 90×g for 10 min at RT, resuspended in 100 uL RT nucleofection working solution and combined with 200 nM miR-629 mimic, inhibitor or scrambled control. Cells/RNA suspension was transferred to a cuvette, placed into the Nucleofector II device, and the U-001 NK program was applied. The cuvette was removed from the device and 500 uL pre-equilibrated culture medium was added. The sample was then transferred to the prepared 12-well plate (final volume approximately 2 mL media/cells per well) and incubated in a humidified 37° C./5% CO2 incubator. As reported herein above, miR-629 was significantly differentially expressed between cell lines having high sensitivity versus low sensitivity to in vitro antibody dependent cellular cytotoxicity with an anti-CD19 antibody (MEDI-551), but not Rituximab. miR-629 (among other miRs) was pre-specified for testing in Phase 1 and Phase 2 clinical trials in B-cell malignancies to assess clinical utility in predicting patient response to anti-CD19 antibody (MEDI-551) treatment. Surprisingly, miR-629 expression differed significantly in baseline blood samples between patients with diffuse large B cell lymphoma that responded or that failed to respond to treatment with an anti-CD19 antibody (MEDI-551). This effect was reproducible in single agent (Ph1) and chemotherapeutic combination studies (Ph2). This effect was not observed with Rituximab. Interestingly, patients with lower levels of miR-629 showed an increased response rate to an anti-CD19 antibody (MEDI-551), but not to Rituximab. This observation may be due, at least in part, to the ability of miR-629 to alter NK cell activation markers either via its presence in exosomes or through other means. These results support a role for miR-629 in mediating response to an anti-CD19 antibody (MEDI-551), but not Rituximab.

### RNA Isolation

Total RNA was extracted from PAXgene blood tubes from lymphoma and leukemia patients or healthy volunteers using the microRNA PAXgene Blood RNA kit (Qiagen, Hilden, Germany). For cell lines and PBMC samples from chronic lymphocytic leukemia patients, samples were isolated using a miRVana miRNA Isolation Kit (Life Technologies) according to the manufacturer's instructions. RNA purity and concentration were determined spectrophotometrically (260/280>1.9). RNA quality was assessed on an Agilent 2100 Bioanalyzer using the RNA 6000 Nano LabChip®.

### TaqMan Q-PCR

For TaqMan analysis, 250-300 ng of total RNA was reverse transcribed to cDNA using Multiscribe RT and miRNA primer pools according to manufacturer's instructions. The resulting cDNA was preamplified using TaqMan PreAmp Master Mix and miRNA primer pools in a reaction containing 12.5 μL 2×TaqMan PreAmp Master Mix, 2.5 μL 10× Megaplex PreAmp primers, 7.5 μL H₂O and 2.5 μL RT product. After cycling, amplified samples were diluted 1:4 in DNA Suspension Buffer (TEKnova, Hollister, Calif.) and held at −20° C. or used immediately for PCR. Real-time PCR on the preamplified material was performed with the BioMark Real-Time PCR System using TaqMan assays specific for miR-629 and the housekeeping reference genes RNU44, U6, U47, and RNU24 (Life Technologies). Cycle threshold (Ct) values above 28 were excluded from calculations. Delta Ct values (ΔCt) were calculated using the mean of the four reference genes (RNU44, U6, U47, and RNU24). In cases where Delta Ct values are used for comparison, it is important to note that Delta Ct is inversely related to expression, such that the higher the Delta Ct value, the lower the miR-629 expression. Fold change values were determined by calculating 2^{-ΔΔCt} using miR-629 expression in healthy volunteers as the control.

### Stable Cell Line Generation

The diffuse large B-cell lymphoma cell line Karpas-422 was transduced with a lentiviral vector over-expressing miR-629 or a scrambled miRNA control (Open Biosystems, Huntsville, Ala.) at an MOI of 2-20. Transduced cells were expanded for 1-2 weeks. Utilizing RFP, cells were sorted by fluorescence-activated cell sorting (FACS) into high miR-629 and low miR-629 populations. Clones were also generated using the limiting dilution method. Over-expression of miR-629 was evaluated by TaqMan QPCR.

### Cell Growth and Apoptosis Assays

miR-629 over-expressing lymphoma cells were treated with 5 μM or 10 μM of etoposide, then cell growth and apoptosis were measured. Cell growth was measured 24 hr and 48 hr post-etoposide treatment with the Cell Titer-Glo Luminescent Cell Viability Assay (Promega, Madison, Wis.) according to the manufacturer's protocol. Caspase activation was measured 48 hr post-etoposide treatment using the Caspase-Glo 3/7 Assay (Promega) according to the manufacturer's protocol. All luminescent data was collected on a SpectraMax M5 plate Reader (Molecular Devices, LLC. Sunnyvale, Calif.).

### Statistical Analyses

microRNA expression fold-change values were analyzed using Welch's t-test or the Mann-Whitney U non-parametric test. p-values of <0.05 were considered significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of selecting therapy for a subject having a B cell malignancy, the method comprising detecting decreased miR-629 expression in a blood sample of the subject relative to a reference level, wherein detection of said decrease selects the subject for anti-CD19 antibody therapy.

2.-4. (canceled)

5. The method of claim 1, wherein the reference level is obtained by
comparing the level of miR-629 expression to the expression level of other microRNAs present in the sample;
determining the range of miR-629 expression in samples obtained from subject's having a B cell malignancy that is not responsive to treatment with an anti-CD19 antibody; or
measuring the level or range of miR-629 expression in a subject or cell line having reduced sensitivity to anti-CD19 antibody treatment, resistant to the anti-proliferative effects of chemotherapy, or resistant to chemotherapy-induced apoptosis.

6. A method of treating a subject selected as having a B cell malignancy responsive to treatment with an anti-CD19 antibody, the method comprising administering to a selected subject an effective amount of an anti-CD19 antibody, wherein the subject is selected by detecting decreased miR-629 expression in a blood sample of the subject relative to a reference level.

7.-9. (canceled)

10. The method of claim 6, wherein the reference level is obtained by
comparing the level of miR-629 expression to the expression level of other microRNAs present in the sample;
determining the range of miR-629 expression in samples obtained from subject's having a B cell malignancy that is not responsive to treatment with an anti-CD19 antibody; or
measuring the level or range of miR-629 expression in a subject or cell line having reduced sensitivity to anti-CD19 antibody treatment, resistant to the anti-proliferative effects of chemotherapy, or resistant to chemotherapy-induced apoptosis.

11. The method of claim 6, wherein the subject has a lymphoma or leukemia of B cell origin.

12. The method of claim 11, wherein the subject has non-Hodgkin's lymphoma, diffuse large B cell lymphoma, follicular lymphoma, mantle cell lymphoma, multiple myeloma, or chronic lymphocytic leukemia

13. The method of claim 6, wherein the blood sample is whole blood, a peripheral blood mononucleated cell (PBMC) sample, serum, or plasma.

14. The method of claim 6, wherein the anti-CD19 antibody is a human, humanized or chimeric antibody.

15. The method of claim 6, wherein the anti-CD19 antibody comprises a VH domain comprising the amino acid sequence of SEQ ID NO: 1.

16. The method of claim 6, wherein the anti-CD19 antibody comprises a VL domain comprising the amino acid sequence of SEQ ID NO: 5.

17. The method of claim 6, wherein the anti-CD19 antibody is MEDI-551.

18. (canceled)

19. The method of claim 6, wherein detection of a decrease in miR-629 identifies the subject as having increased activation of a natural killer cell.

20. (canceled)

21. The method of claim 6, wherein selection of the subject further comprises detecting the level of expression of a natural killer cell protein selected from the group consisting of granzyme B (GZMB), GZMA, GZMM, cathepsin D, perforin 1, interferon regulatory factor 7, CD63, CD96, NKp30, NKG2D, CD56, and CD107a or a polynucleotide encoding said protein.

22. A kit comprising a primer or probe that specifically binds miR-629.

23. A kit comprising an anti-CD19 antibody and the primer or probe of claim 22.

24.-32. (canceled)

33. The method of claim 1, wherein the subject has a lymphoma or leukemia of B cell origin.

34. The method of claim 1, wherein the anti-CD19 antibody comprises a VH domain comprising the amino acid sequence of SEQ ID NO: 1.

35. The method of claim 1, wherein the anti-CD19 antibody comprises a VL domain comprising the amino acid sequence of SEQ ID NO: 5.

36. The method of claim 1, wherein the anti-CD19 antibody is MEDI-551.

37. The method of claim 1, wherein detection of a decrease in miR-629 identifies the subject as having increased activation of a natural killer cell.
